(19) United States
(12) Patent Application Publication
(10) Pub. No.: US 2020/0234826 A1
(43) Pub. Date: Jul. 23, 2020

(54) PROVIDING PERSONALIZED HEALTH CARE INFORMATION AND TREATMENT RECOMMENDATIONS

(71) Applicant: Outcomes4Me Inc., Cambridge, MA (US)

(72) Inventor: Maya R. Said, Cambridge, MA (US)

(21) Appl. No.: 16/711,164

(22) Filed: Dec. 11, 2019

Related U.S. Application Data
(60) Provisional application No. 62/778,018, filed on Dec. 11, 2018.

(51) Int. Cl. G16H 50/20; G16H 10/60; G16H 10/20

(52) U.S. Cl. CPC G16H 50/20; G16H 10/20; G16H 10/60

(57) ABSTRACT

Various embodiments described herein provide mechanisms for implementing an integrated platform that provides personalized health care information and treatment recommendations for patients and caregivers. Retrieved patient data are used as input into a personalization algorithm that generates specific, personalized treatment recommendations and options for the patient. Treatment recommendations and options are displayed to the patient in ordinary language to facilitate proper understanding. Translation of treatment options to ordinary language can be performed automatically.

### CROSS-REFERENCE TO RELATED APPLICATION

The present application claims the benefit of U.S. Provisional Application Ser. No. 62/778,018 for “Integrated Platform for Providing Personalized Health Care Information and Verified Social Network for Patients” (Attorney Docket No. OUT001-PROV), filed Dec. 11, 2018, which is incorporated by reference herein in its entirety.

### TECHNICAL FIELD

The present document relates to mechanisms for enabling communications between patients and healthcare providers.

### BACKGROUND

Patients diagnosed with certain diseases are often given multiple alternative treatment options from which to choose. However, the number of options provided to a patient, and the information on these options, are inconsistent and often depend on the physician and health care provider, their geographic locations, and/or other factors.

Various resources exist to provide information on available treatment options for a given disease. However, such existing resources generally do not provide tools that a patient and his or her caregiver can use to automatically get treatment options specific to the patient's diagnosis and clinical and treatment history. Rather, the information provided by such resources is usually generally applicable to a given disease and various clinical/treatment history cases.

Furthermore, patients and their caregivers often lack detailed diagnostic information and treatment histories that may be available to the doctor or hospital. Thus, in spite the abundance of information on treatments and treatment options, existing resources available to patients and their caregivers often fail to provide the tools to inform patients adequately about their treatment options, and further fail to provide real alternatives that patients can discuss with their doctors. Such existing resources also often lack the ability to effectively and efficiently receive patient data and/or requests for summary data, and further lack the ability to display the resulting information in a manner that facilitates proper understanding by the patient.

Although some hospital systems have their own patient portals that can be used to display health information to patients, such systems do not provide mechanisms for retrieving and merging health records from different systems and institutions for display to the patient in an integrated manner. In addition, such systems do not generally provide ways for data to be collected directly from patients and then displayed in summary form for patients and/or providers.

### SUMMARY

The present document relates to techniques for implementing an integrated platform that provides personalized health care information and treatment recommendations for patients and caregivers. The techniques described herein can be implemented singly, or in any suitable combination with one another.

In various embodiments, the automated techniques described herein provide personalized treatment information to patients and their caregivers without the need for patients to search for the treatment or talk to a doctor. According to various embodiments, patient data can be directly retrieved from health care systems and automatically analyzed.

Examples of patient data include but are not limited to electronic medical records, genomic information, claims records, geolocation, diagnostic tests, medical benefits, insurance plan information, and the like. Such data can be collected from any suitable source, including for example active and/or passive trackers such as FitBit devices, smart watches, heart rate monitors, and/or the like. Examples of health care systems include but are not limited to hospital systems, physician clinics, payers systems, diagnostic companies, clinical labs, and the like.

Retrieved patient data can be used as input into a personalization algorithm that generates specific, personalized treatment recommendations and options for the patient. Treatment recommendations and options are displayed to the patient in ordinary language to facilitate proper understanding. In at least one embodiment, the translation of treatment options to ordinary language can be performed automatically. Treatment recommendations and options include but are not limited to: approved drugs, interventions such as surgery or radiation therapy, and clinical trials.

In at least one embodiment, the system is implemented as a software platform and mobile application that helps patients and caregivers navigate their treatment options, and connect them with available treatment opportunities, including standard-of-care options and clinical trials. By delivering a personalized and expert-validated evidence-based experience, the tools described herein can enhance care quality and effectiveness, as well as allow care delivery beyond clinical walls into the home setting. This improves patient outcomes and accelerates innovation through more comprehensive patient follow-up.

In at least one embodiment, the patient-centric experience provided by the described system is updated based on feedback from patients, caregivers, and providers, thus ensuring that the system evolves as the needs of patients and caregivers evolve.

In at least one embodiment, the system provides functionality for real-time data capture and symptom reporting.

Particular examples, applications, and variations are described in more detail below.

### BRIEF DESCRIPTION OF THE DRAWINGS

The accompanying drawings, together with the description, illustrate several embodiments. One skilled in the art will recognize that the particular embodiments illustrated in the drawings are merely exemplary, and are not intended to limit scope.

FIG. 1 is a block diagram depicting an architecture for a system that provides personalized treatment recommendations to patients, according to one embodiment.

FIG. 2 is a flow diagram depicting a treatment personalization method according to one embodiment.

FIGS. 3A to 3I depict various examples of screens of a user interface for medical records workflow according to one embodiment.

FIGS. 4A and 4B depict various examples of screens of a diagnosis update according to one embodiment.

FIGS. 5A to 5F depict various examples of screens of a user interface for a treatment path according to one embodiment.

FIG. 5G depicts a portion of a treatment personalization algorithm that can be used to populate treatment options screens, according to one embodiment.

FIGS. 6A to 6C depict various examples of screens of a user interface for clinical trials workflow including GPS functionality, according to one embodiment.

FIG. 7A depicts an example of a screen of a user interface for applying outcomes filters to find treatments, according to one embodiment.

FIG. 7B depicts an example of a screen of a user interface for presenting a visualization of patent outcomes for a particular treatment and for receiving a request to be privately matched with another patient for anonymous communication, according to one embodiment.

FIG. 7C depicts examples of screens of a user interface for presenting drug-level outcomes data, according to one embodiment.

FIG. 8 depicts an example of a screen of a user interface for presenting treatment options, sorted by costs, and for presenting treatment details including personalized, benefit-specific patient costs, according to one embodiment.

FIGS. 9A and 9B depict an example of a treatment personalization algorithm for metastatic breast cancer, according to one embodiment.

FIGS. 10A through 10F depict an example of an algorithm for personalized screening according to one embodiment.

FIG. 11 is a block diagram depicting an overall schematic architecture for a system that provides personalized treatment recommendations to patients, according to one embodiment.

FIG. 12 depicts an example of an application of a personalization engine to generate appropriate treatment options for a patient, according to one embodiment.

FIG. 13 depicts an example of a patient-reported outcome (PRO) service, according to one embodiment.

FIG. 14A is a block diagram depicting an example of integration of apps into existing electronic health record (EHR) systems and workflows, according to one embodiment.

FIG. 14B is a block diagram depicting relationships between a Secure Care Plan and other entities, according to one embodiment.

FIG. 15 is a flow diagram depicting a method of integrating the described system into existing electronic health record (EHR) systems and workflows, according to one embodiment.

FIGS. 16A through 16C depict various examples of screens of a user interface for patients to self-report symptoms and quality-of-life data, according to one embodiment.

FIGS. 17A and 17B depict various examples of screens of a user interface for completing a Patient Record Outcomes Questionnaire, according to one embodiment.

### DETAILED DESCRIPTION OF THE EMBODIMENTS

The systems and methods set forth herein may be applied to many contexts in which personalized information and/or recommendations are to be exchanged between and among a patient and various health care providers. For illustrative purposes, the description herein is set forth with respect to a method and system wherein such communication takes place via the Internet and/or wireless communications devices such as smartphones. One skilled in the art will recognize that the systems and methods described herein may be implemented in a wide variety of other contexts, and using other hardware and/or software arrangements. Therefore, the particular techniques described herein are provided merely for illustrative purposes.

### System Architecture

According to various embodiments, the systems and methods described herein can be implemented on any electronic device or set of interconnected electronic devices, each equipped to receive, store, and present information. Each electronic device may be, for example, a computing device, desktop computer, laptop computer, smartphone, voice-enabled device, smart speaker, server, tablet computer, smart headphones, Internet-connected device, and/or the like. As described herein, some devices used in connection with the system described herein are designated as client devices, which are generally operated by end users. Other devices are designated as servers, which generally conduct back-end operations and communicate with client devices (and/or with other servers and/or other components) via a communications network such as the Internet. In at least one embodiment, the methods described herein can be implemented in a cloud computing environment using techniques that are known to those skilled in the art.

In addition, one skilled in the art will recognize that the techniques described herein can be implemented in other contexts, and indeed in any suitable device, set of devices, or system capable of interfacing with existing data storage systems and/or e-commerce systems. Accordingly, the following description is intended to illustrate various embodiments by way of example, rather than to limit scope.

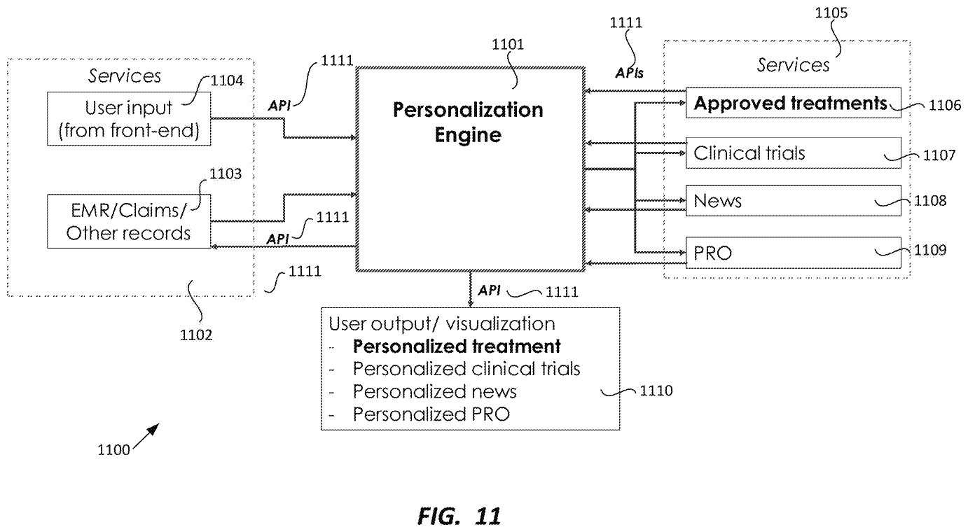

Referring now to FIG. 11, there is shown a block diagram depicting an overall schematic architecture for a system 1100 that provides personalized treatment recommendations to patients, according to one embodiment. Personalization engine 1101 takes input from various sources, including services 1102 and 1105, to generate user output 1110, which may include personalized treatment, clinical trials, news, and/or patient-reported outcomes (PRO). In at least one embodiment, input to personalization engine 1101 can be provided via various application programming interfaces (APIs) 1111. Services 1102 can provide information from user input 1104, collected via an app or other software, and/or information from medical records, claims, and/or the like 1103. Services 1105 can provide information regarding approved treatments 1106, clinical trials 1107, news 1108, and patient-reported outcomes (PRO) 1109.

In at least one embodiment, the data collected about a patient can include patient-generated data, such as patient-reported outcomes data and/or data from sensors such as FitBit devices, smart watches, heart monitors, and/or the like. In at least one embodiment, such data is used to recognize similarities among patients (for example, via a distance metric), and then use these similarities to generate recommendations for resources, treatments, and/or the like. Such similarities can also be used to generate suggestions for social media contacts. The system can then provide mechanisms for enabling anonymous communication, such as via a social network, among patients with similar issues and/or conditions.

In at least one embodiment, the system is implemented on a platform that consists of a mobile application, API servers, databases, and internal administrative tools. In at least one embodiment, the platform is a web services architecture constructed using standard tools such as EC2, PostgreSQL and Node.js. The system can use API integrations with a number of third-party systems such as Wolters Kluwer Health and NCI, among others.

In at least one embodiment, all aspects of the system and platform comply with all applicable laws and regulations, including the Health Insurance Portability and Accountability Act of 1996 (HIPAA). The system also provides industry-standard security to ensure data privacy. In particular, in at least one embodiment:
No data is stored on mobile devices.
All cloud services are HIPAA-compliant services, for example operated by Amazon Web Services (AWS) with Backend-as-a-service (BaaS) in place.
All transmitted data (data in motion) is encrypted.
All stored data (data at rest) is encrypted.
Access to identified patient health information is secure and restricted.
All access to identified patient health information is logged.
No patient health information is stored outside of the secure cloud-based platform.

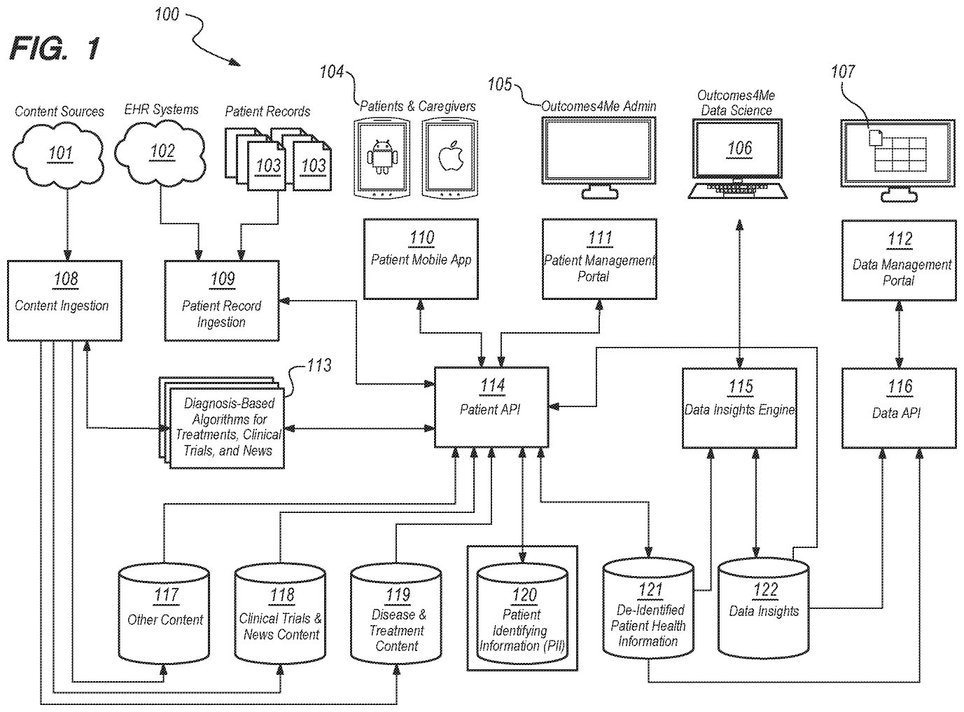

Referring now to FIG. 1, there is shown a block diagram depicting an example of a more detailed architecture for a system 100 that provides personalized treatment recommendations to patients, according to one embodiment. One skilled in the art will recognize that the various components and arrangement of such components as depicted in FIG. 1 is merely exemplary, and that the techniques described herein can be implemented using other components and/or arrangements. In at least one embodiment, the architecture shown in FIG. 1 can implement functionality depicted in the schematic architecture of FIG. 11.

In some embodiments, one or more components, as shown and described herein in connection with FIG. 1, may be used to implement the system and method described herein. Such components may be implemented in a cloud computing-based client/server architecture, using, for example, Amazon Web Services, an on-demand cloud computing platform available from Amazon.com, Inc. of Seattle, Wash. Therefore, for illustrative purposes, the system and method are described herein in the context of such an architecture. One skilled in the art will recognize, however, that the system and method can be implemented using other architectures, such as for example a stand-alone computing device or distributed computing architecture, rather than a client/server architecture.

Further, the functions and/or method steps set forth below may be carried out by software running on one or more of the various devices and components depicted in FIG. 1. This software may optionally be multi-function software that is used to retrieve, store, manipulate, and/or otherwise use data stored in data storage devices 117-122 and/or other components.

In the context of the description herein, a “user” may be a patient, health care provider, assistant, administrator, system administrator, manager, consumer, prospective consumer, customer, and/or any other individual, enterprise, or other group, which may optionally include one or more users. Data storage devices 117-122 may be implemented using any data store or other storage device, which may include any device capable of digital data storage, including any known hardware for nonvolatile and/or volatile data storage, and may be implemented using known database techniques. A collection of such data stores may form a “data storage system” that can be accessed by multiple users.

In the context of the description herein, a “computing device” or “client device” may be any device capable of digital data processing, including (but not limited to) one that is able to receive any combination of text-based input, touchscreen input, voice-based (speech) input, and/or the like, and is further able to generate any combination of visual output, text-based output, haptic output, and/or audio output (including voice-based (speech) output). As described below, the various hardware components set forth herein may communicate with one another via any suitable electronic network, which may include wired and/or wireless components.

In the context of the description herein, a “server” may be any computing device that performs back-end processing and/or functionality for implementing the techniques described herein. Such a device may communicate as needed with client devices, networking components, and/or storage devices, to implement the techniques described herein.

FIG. 1 depicts various data storage elements 117-122. One skilled in the art will recognize that these may represent any number of separate storage devices, or their functionality may be combined into one or more physical storage devices. Storage elements 117-122 thus need not correspond on a one-to-one basis with physical storage devices. Storage elements 117-122 may be provided at a single location or at locations that are separated from one another. Any known data storage technology can be used to implement storage elements 117-122. In various embodiments, storage elements 117-122 can be implemented as databases using any known database technology, or they can be implemented in other ways, or any combination thereof.

In at least one embodiment, the following data storage elements are provided:
Other content 117: Stores any additional content needed or used by the system. Populated by content ingestion component 108; used by patient API 114.
Clinical trials and news content 118: Stores data describing current and/or past clinical trials, and/or any relevant news events. Populated by content ingestion component 108; used by patient API 114. In at least one embodiment, separate data storage elements can be provided for clinical trials and news content, respectively.
Disease and treatment content 119: Stores data describing diseases and treatment. Populated by content ingestion component 108; used by patient API 114.
Patient identifying information (PII) 120: Patient information that includes identifying information. This may include sensitive and/or confidential data; therefore, in at least one embodiment it is securely stored and requires appropriate authentication to access, in compliance with applicable laws and regulations. Populated by and used by patient API 114.
De-identified patient health information 121: Patient information that does not include identifying information. This may be used for data analytics and/or data insights. Takes input from and interfaces with patient API 114. Used by data insights engine 115 and data API 116.
Data insights 122: Contains data insights generated by data insights engine 115 using data from de-identified patient health information 121. Takes input from and interfaces with patient API 114 and data insights engine 115. Used by data API 116.
In at least one embodiment, additional data storage elements can also be provided, such as, for example, for resources.

The other components shown in FIG. 1 perform various other functions, as follows.

In at least one embodiment, content ingestion component 108 retrieves relevant content from various content sources 101, which can include any number of public and private third-party systems. Component 108 can also communicate with diagnosis-based algorithms 113, described below, to collect content generated by algorithms 113.

In at least one embodiment, the retrieved content is normalized and categorized so that it may later be provided only to the proper audience by the personalization platform described herein. In at least one embodiment, content ingestion component 108 stores content in any number of suitable data storage devices, including for example other content storage 117, clinical trials and news content storage 118, and/or disease and treatment content storage 119. In at least one embodiment, content stored in storage devices 117-119 can be stored using any known database storage techniques.

In at least one embodiment, patient record ingestion component 109 collects health information of patients from sources such as electronic health record (EHR) systems 102 and/or patient records 103. In at least one embodiment, such collection is performed using appropriate secure data connections so as to ensure patient confidentiality and to conform to applicable laws and regulations. In at least one embodiment, patient record ingestion component 109 provides process tools for the parsing, annotation, and incorporation of collected health information into the personalization platform described herein. In at least one embodiment, patient record ingestion component 109 communicates with patient application programming interface (API) 114 to enable such functionality.

In at least one embodiment, diagnosis-based algorithms 113 are provided for treatments, clinical trials and news. Such algorithms 113 can run on any suitable server, cloud computing platform, or other computing component. In at least one embodiment, algorithms 113 are a collection of rigorously refined and tested processes for delivering individually personalized results to patient users. In at least one embodiment, algorithms 113 take input from content ingestion component 108 and provide results to patient API 114.

In at least one embodiment, patient API 114 is provided, to interface with various components of the system, provide functionality, and ensure that all of the various services and components of the platform work in concert with one another.

In at least one embodiment, patient mobile app 110 runs on device(s) 104 associated with patients and/or caregivers. Patient mobile app 110 interfaces with patient API 114 as needed, so as to provide a user-friendly interface that allows access to tools for patients and/or caregivers to develop individual patient profiles. The system then uses these patient profiles to provide specifically-tailored content and experiences appropriate for a particular user. In at least one embodiment, additional apps may also be provided, such as an app that provides a dashboard for healthcare providers to create, review, and update protocols and other settings. In at least one embodiment, other user-facing technologies and interfaces can be provided in addition to or instead of apps (including patient mobile app 110), such as for example, kiosks, computer interfaces, web pages, and/or the like.

In at least one embodiment, the system includes patient management portal 111 as an internal tool to allow management and operations staff to provide service and support to patient users in a secure and private manner.

In at least one embodiment, the system includes data insights engine 115 as an internal tool that provides analytical access to management and operations staff, without exposing or compromising the privacy of patient users.

In at least one embodiment, data management portal 112 is a customer-facing tool that allows data customers to perform analyses on datasets to which they subscribe. “Data customers” include those entities that may be interested in analytics and other insights into the data collected by the system. In at least one embodiment, such data customers can access data management portal 112 via their own client devices 107. Relevant reports, graphics, and/or interactive applications can be presented by data management portal 112 on such devices 107.

In at least one embodiment, data API 116 provides controlled access to data customers. In at least one embodiment, data API takes data from various data storage devices, such as de-identified patient health information 121 and data insights 122, and processes such data for presentation via data management portal 112. In at least one embodiment, data API 116 allows data customers direct-to-API access should they desire it.

Referring again to FIG. 11, in at least one embodiment, the various components depicted therein can be implemented using any combination of components depicted in FIG. 1. For example, personalization engine 1101 may be implemented on patient API 114 and patient mobile app 110. Services 1102, 1105 may obtain information from data storage 117-122. User output 1110 may be provided via patient mobile app 110.

In at least one embodiment, the system retrieves and uses information from electronic health records (EHR) and/or other sources, as input for generating personalized information and treatment recommendations.

In many cases, various healthcare providers may be using different healthcare systems that in turn use different EHR vendors and workflows. Conventionally, creating direct integration between these various systems can be difficult and costly, and in many cases infeasible given competing business needs and priorities.

Accordingly, in at least one embodiment, patient mobile app 110 is used as a hub that enables extraction and integration of data to/from various EHR systems and workflows. A provider-facing Secure Care Plan (SCP) dashboard is provided, which enables care providers across various healthcare systems to review and update the SCP. In at least one embodiment, the Secure Care Plan can include a Survivorship Care Plan, although in other embodiments it can incorporate other elements beyond survivorship.

In at least one embodiment, the system employs a universal open API, thereby ensuring rapid diffusion across its various components. So as to maintain compatibility with most existing EHR systems, the system may use the Substitutable Medical Applications and Reusable Technologies (SMART) platform, operation on the Fast Healthcare Interoperability Resources (FHIR) API (SMART on FHIR).

FHIR is a “next generation” interoperability standard developed by HL7, a not-for-profit, ANSI-accredited standards developing organization. It incorporates many concepts already familiar to developers from other domains such as Resources to represent common healthcare concepts such as Medications and Problems and a simple REST-based API made popular by some of the major internet players such as Google, Twitter and Facebook. All the major EHR vendors have collaborated over the interpretation and profiling of the FHIR standard through the Argonaut Project.

SMART adds a layer of security in front of FHIR interfaces by leveraging OAuth2 for Authentication and Authorization, and OpenID Connect for user Identity. SMART also standardizes the process of negotiating access to information and operations between client and server. It also describes a process by which an EHR application can launch an external application preserving context, and provide safe access to the data within the EHR. EHR vendors have incorporated support for SMART into their products and are rolling out the technology to health care institutions. Many leading EHR companies, and other technology companies, have built SMART support into their products. Hundreds of hospitals and health systems offer this capability.

In at least one embodiment, the system exchanges data between and among various EHRs and workflows via a SCP. In at least one embodiment, certain individuals may be able to access and edit the SCP, such as for example:
A physician, such as a primary care physician, specialist (such as an oncologist), or advanced practice provider at an institution having an EHR system; via a provider-facing dashboard.
A patient or patient navigator (PN); via a patient/PN-facing mobile app, using his or her own credentials.

In at least one embodiment, the SCP provides a date/time stamp, and is able to synchronize with the EHR system(s) of any number of physicians caring for the patient, even if they are using different healthcare systems.

In at least one embodiment, the system integrates into any existing EHR system and workflows using a SMART on FHIR approach. For example, the system can integrate with systems that use EPIC as the EHR vendor. In at least one embodiment, two levels of integration are employed to deliver the functionality described herein:
Provider-level: Connects the provider-controlled SCP dashboard to the EHR, preserving context, and providing safe access to the data within the EHR with the ability to:
   Read from the EHR vendor (such as EPIC) the diagnostic, treatment and scheduling information needed to create and update the SCP
   Write into the EHR vendor the SCP and its updates
Patient-level: Connects the patient/PN-facing mobile app to the EHR with the ability to:
   Read the relevant data from the patient's chart
   Write into the EHR vendor the SCP updates

The system ensures full compatibility with existing IT infrastructure, including systems interoperability (for example to other providers using Cerner or other), cybersecurity, and patient privacy requirements.

By using a SMART on FHIR approach, the system ensures that subsequent installations with other healthcare systems are streamlined, requiring significantly less resource allocation from the hospital's IT group, and simplifying project management. Such an approach also ensures that integration of the system into existing architectures and EHR systems is standardized, requiring little to no custom development. Another benefit is a consistent user and patient experience across EHR platforms.

In at least one embodiment, the system is implemented using a web-based architecture that employs standard tools such as Node.js and cloud-based HIPAA-compliant infrastructure hosted by AWS. Appropriate security and privacy protocols are employed to further ensure HIPAA compliance and a high level of security.

In at least one embodiment, the system includes multiple different apps that integrate with existing EHR systems and existing workflows. These can include, for example:
a provider dashboard to create, review and update the SCP; and
patient/PN-facing mobile app 110 that includes the SCP and additional features to facilitate patient navigation through survivorship care.

To ensure maximum value to patients and providers, in at least one embodiment, all provided apps integrate into all existing EHR systems and workflows.

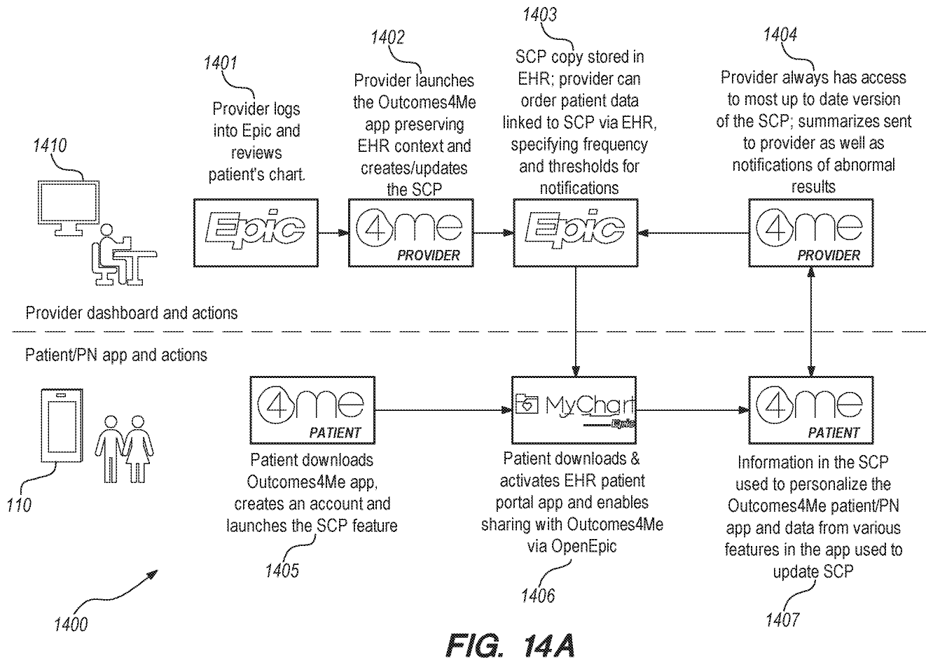

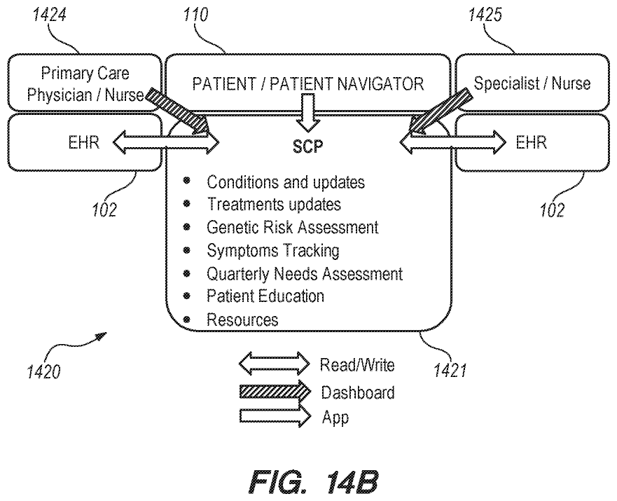

Referring now to FIG. 14A, there is shown an example 1400 of such integration, according to one embodiment. The top part of example 1400 depicts steps associated with provider dashboard 1410. In step 1401, the healthcare provider logs into an existing EHR system (such as EPIC), and reviews the patient's chart. In step 1402, the provider launches provider app 1410; app 1410 preserves the EHR context and creates/updates the SCP. In step 1403, an SCP copy is stored in the EHR system; the provider can order patient data, linked to the SCP via the EHR system, specifying frequency and thresholds for automated notifications. Step 1404 illustrates that the provider always has access to the most up-to-date version of the SCP; summaries are sent to the provider, and the provider is automatically notified of any abnormal results.

The bottom part of example 1400 depicts steps associated with patient app 110. In step 1405, the patient downloads app 110, creates an account, and launches the SCP feature. In step 1406, the patient downloads and activates an EHR patient portal app, and enables sharing via any suitable platform, such as OpenEpic. In step 1407 information in the SCP is used to personalize app 110, and data from various features in app 110 is used to update the SCP.

Referring now to FIG. 14B, there is shown a block diagram depicting relationships between a Secure Care Plan 1421 and other entities, according to one embodiment. In at least one embodiment, SCP 1421 is used as the repository for truth and accuracy. By synchronizing the data in SCP 1421 with other entities that may store and/or use patient information, such as EHR systems 102, mobile app 110, primary care physician/nurse 1424, and/or specialist/nurse 1425, the system ensures compatibility with all external systems. The system thereby eliminates the need for two different EHR systems 102 to directly talk to each other and synchronize.

### Integration

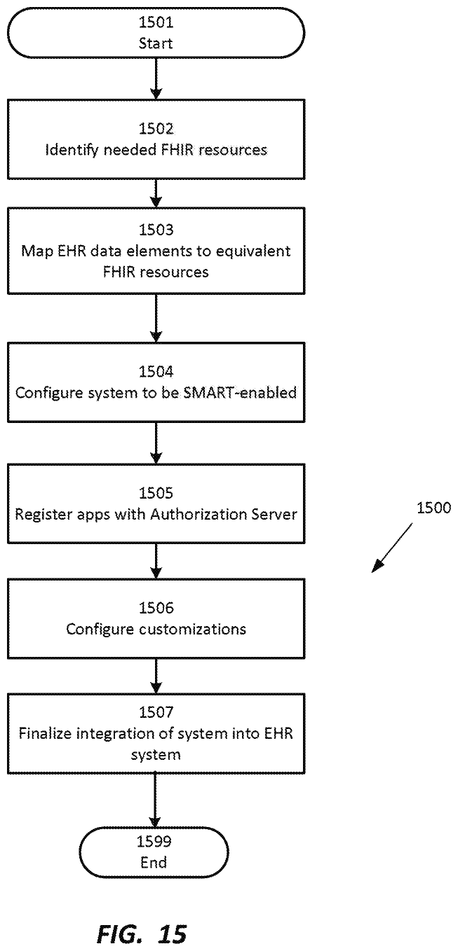

Referring now to FIG. 15, there is shown a flow diagram depicting a method 1500 of integrating the described system into existing electronic health record (EHR) systems and workflows, according to one embodiment.

The method begins 1501. Needed FHIR resources are identified 1502. EHR data elements are then mapped 1503 to the equivalent FHIR resources.

In at least one embodiment, steps 1502 and 1503 are done at the outset of the project and take into account the design of the SCP. For example, the following FHIR resources might be identified and used: Patient (name, gender, date of birth, demographics); Practitioner; MedicationOrder; MedicationStatement; Condition; Observation; Diagnostic Report; Procedure; CarePlan; and Goal.

The system is then configured 1504 to be SMART-enabled, and the apps (including, for example patient mobile app 110 and provider app 1410) are registered 1505 with an Authorization Server. This can involve development using the SMART sandbox and other available data. In at least one embodiment, various approaches can be tested before full deployment. In particular, the patient mobile app 110 can provide test patient data, and the FHIR browser can facilitate review of changes to the data made by patient mobile app 110. In at least one embodiment, a SMART launcher can be used to configure, test, and demonstrate mobile app 110 using sample patients in the SMART sandbox.

Appropriate customizations can then be configured 1506. For example, if the system is being configured for use with a particular EHR system 102 such as EPIC, EPIC-specific customizations can be performed. EPIC offers a variety of resources to meet FHIR integration needs. The system can therefore be configured based on a determination of which resources are best suited for a particular application. For example, open.epic provides free resources to support patient-controlled apps (such as app 110) that access the common clinical data set (CCDS) from the patient's chart. Epic USCDI on FHIR is geared towards provider-controlled applications and also offers free resources to access the U.S. Core Data for Interoperability (USCDI). Finally, the Epic App Orchard developer program provides additional resources including access to additional Epic APIs (FHIR or otherwise) to create further integration with EPIC. In at least one embodiment, an EPIC-provided sandbox is used to test and validate app 110 before deploying it into a live system.

Next, the integration of the system into EHR system 102 (such as EPIC) is finalized 1507. Any of several methods are available to retrieve needed FHIR resources for this process, including for example:
EPIC web services. The preferred method when possible is to use EPIC's provided web services to retrieve data elements, then expose them via the FHIR interface. The advantage is that these web services take care of the required auditing and logging with minimal overhead. They are also fast and efficient, and pull data from Chronicles, EPIC's real-time clinical database.
Clarity. When there is not a web service available for a specific data type, the data can be quickly and easily pulled from Clarity, a relational database that is updated daily with data from the production clinical database. This may not be ideal for data elements that require real-time accuracy, but can be useful for prototyping FHIR resources.
Shadow. When a web service is not available, the system can use Shadow, the reporting database that is synchronized with Chronicles, EPIC's real-time clinical database. The database can use the Caché database management system based on the Massachusetts General Hospital Utility Multi-Programming System, or MUMPS.

The method then ends 1599.

In addition to mapping the EHR data elements to the appropriate FHIR resources, a critical element to full workflow integration is the authentication and authorization process. In at least one embodiment, an integrated process such as single sign-on (SSO) is employed so that providers are not required to log in to the SCP dashboard after they have already logged into their EHR environment. This integration can be accomplished through the standard OAuth 2.0 standard, and may require modification of the EHR environment so that the EHR generates a token that can then be understood and interpreted by the FHIR infrastructure, which in turn grants appropriate access to an authorized user. In at least one embodiment, a web-based view of the patient's SCP within Hyperspace is integrated using single sign-on and patient context secure launch of SCP. This allows a health care provider, such as a physician or specialist, to interact with patient SCP information directly from the SCP system without relying solely on the data integrated into EPIC.

In at least one embodiment, workflow integration is also performed, for example by integrating the system into provider and patient navigators' workflows. For example, the system can determine where within the clinical workflow apps 110, dashboard app 1410 should be made available, and make it simple to invoke app 110 or app 1410 within that workflow. Examples include adding a button to the same menu as the EHR existing care plan functionality. When clicked or tapped, it opens provider-facing SCP dashboard app 1410 in the same manner as the default care plan app. In addition, patient mobile app 110 can be integrated directly into existing software functionality, such as a myChart portal, so that it can be invoked by activating a single button without any additional authentication.

In other embodiments, integration can be performed using other approaches. These include, for example, HL7 interfacing, custom flat-file extracts out of EHR systems 102 such as EPIC, and/or custom document submission feeds into EHR systems 102. In the event that extensive customization is needed on the healthcare system side, the system can employ third-party solutions such as those available from Redox, Inc. of Madison, Wis., in which an authorization layer is implemented at the system level through interactions with the businesses involved. Redox completes the setup process and verifies who is authorized to receive data. As a result, Redox supports apps regardless of EHR or language and the ability to read and write.

### Personalization Method

As mentioned above, the automated techniques described herein provide personalized treatment information to patients and their caregivers without the need for patients to search for the treatment or talk to a doctor. According to various embodiments, patient data can be directly retrieved from health care systems, automatically analyzed, and used for presenting relevant, personalized information to patients and their caregivers, and for connecting them to one another.

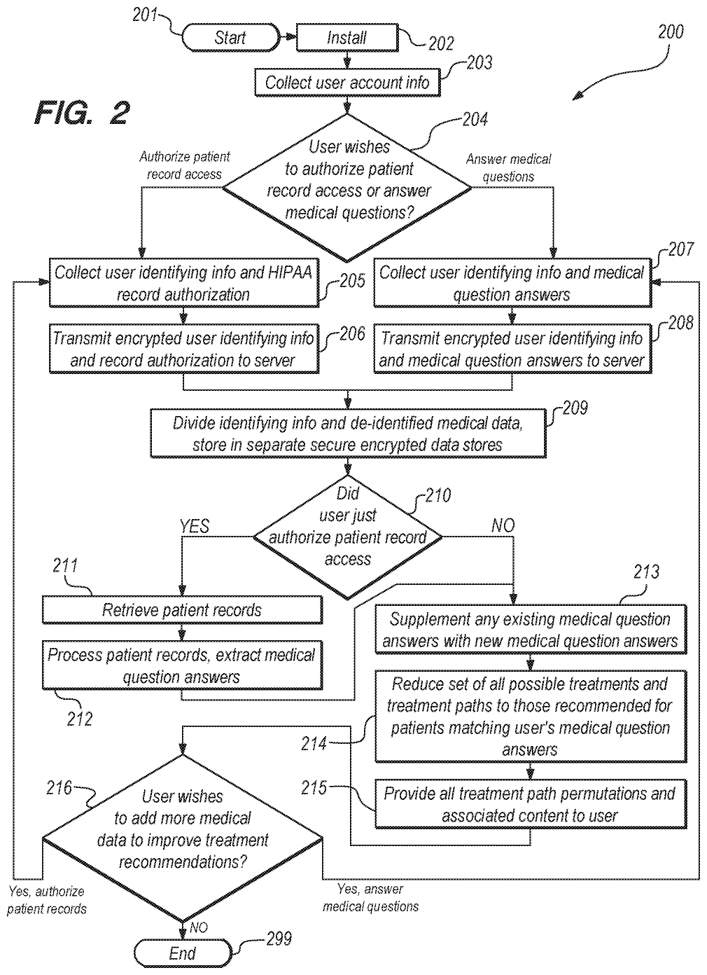

Referring now to FIG. 2, there is shown a flow diagram depicting a treatment personalization method 200 according to one embodiment. In at least one embodiment, the method of FIG. 2 can be implemented using a system architecture such as that shown in FIG. 1. For example, in at least one embodiment, method 200 is implemented on components of FIG. 1 such as patient API 114, patient mobile app 110, and/or diagnosis-based algorithms component 113. Accordingly, for illustrative purposes, several of the steps of FIG. 2 are described as presenting information and/or collecting information via patient mobile app 110. However, one skilled in the art will recognize that the method of FIG. 2 can be implemented on other systems having different arrangements and/or components.

In various embodiments, information can be personalized based on patient information that is automatically retrieved from authorized patient records, and/or user answers to questions, for example via a questionnaire. In at least one embodiment, personalization is performed by automatically looking up specific treatments based on patient attributes.

In at least one embodiment, users download and install mobile app 110 prior to first use. As described in more detail below, upon login, patients are given two options to enter clinical information regarding their disease and treatment: Patients can enter clinical information based on the best of their knowledge, or they can allow access to their medical records by signing a patient-directed HIPAA release request. The specific clinical information of interest will be then extracted directly from the retrieved medical records.

Data to be entered or accessed can include disease-specific diagnostic and treatment information, such as for example: HER-2 and hormone status; whether the patient has had surgery or not vs. metastatic disease; whether the patient has started treatment or not; and what type of treatment the patient has received.

As described in more detail below, the system then generates personalized information and/or treatment recommendations, based on the provided clinical information. Such personalized information and recommendations can include, for example:
the next standard of care treatment options for the patient according to appropriate guidelines (such as for example National Comprehensive Cancer Network (NCCN) guidelines);
clinical trials matching the patient's disease status and desired location;
customized news feeds;
customized resources, such as transportation assistance, financial aid, specialized trainers, and/or the like; and/or
information about patients who have similar clinical histories and preferences, and/or who have responded in a specific way to treatments.

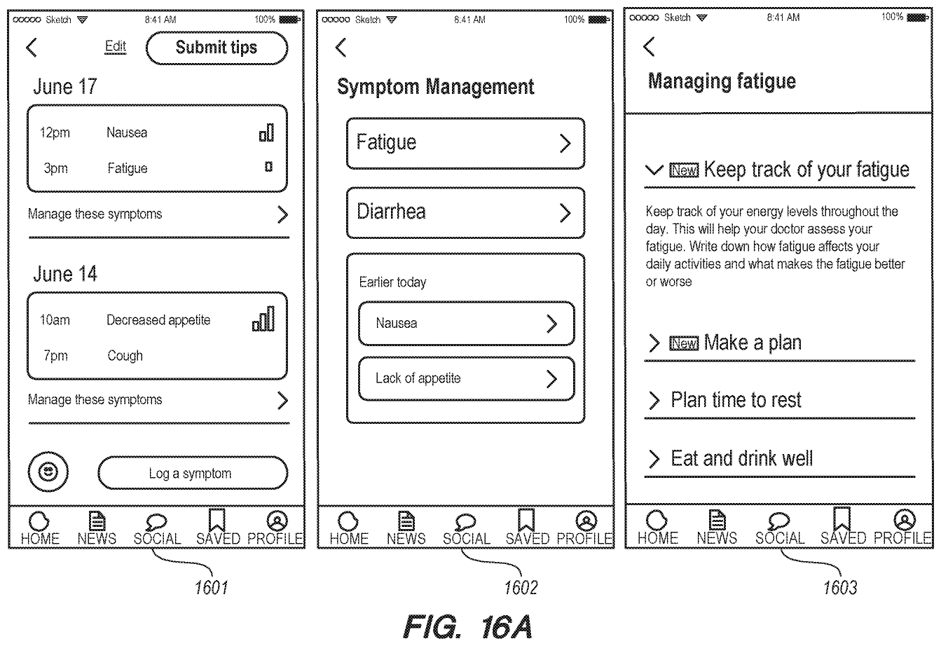

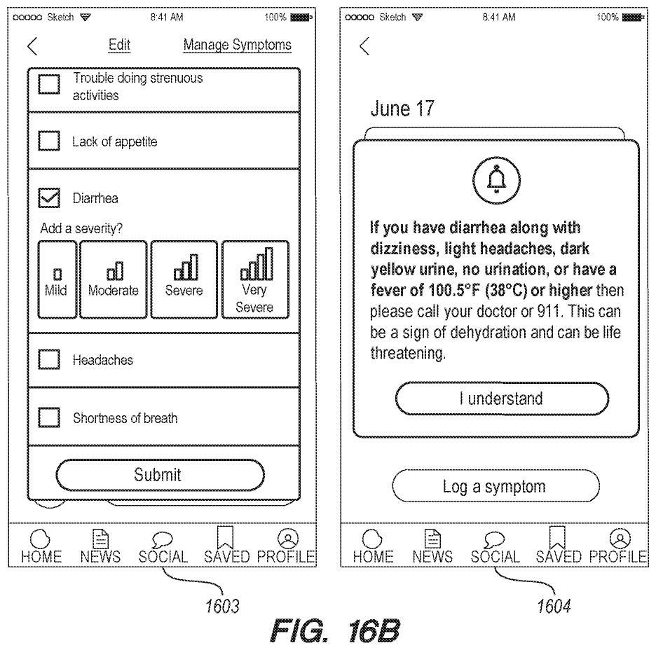

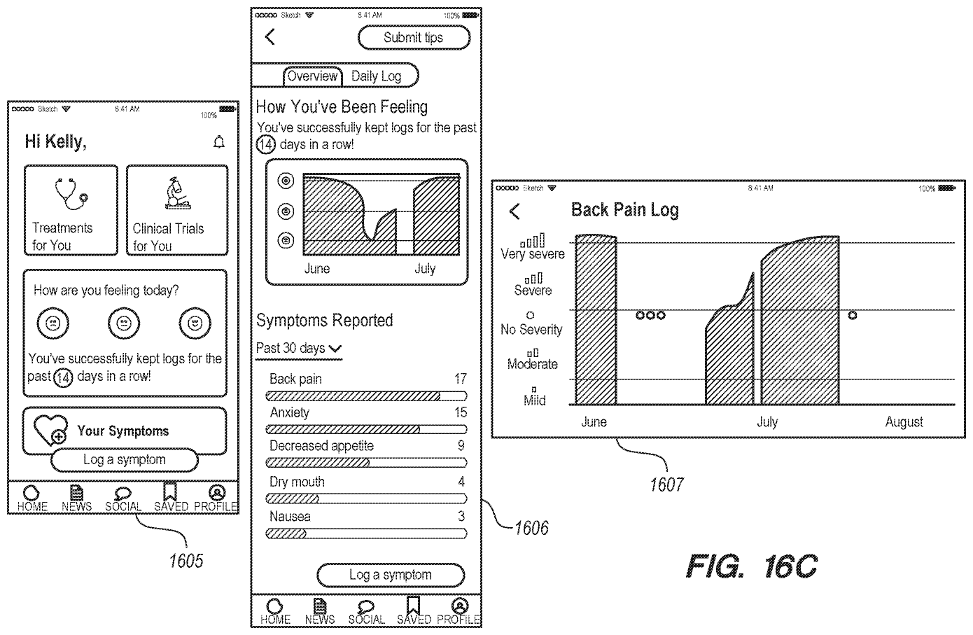

Patients are also able to self-report quality of life data and their symptoms. Referring now to FIGS. 16A through 16C, there are shown various examples of screens of a user interface for patients to provide such information, according to one embodiment.

Screens 1601, 1602, and 1603 provide tips for symptom management. Screens 1603 and 1604 display alerts for severe symptoms. Screen 1605 provides a user interface for self-reporting symptoms. Screens 1606 and 1607 provide displays of previously reported symptoms.

In at least one embodiment, the system can collect patient information from sensors, such as those on a wearable device such as a FitBit device or smart watch (such as, for example, an Apple Watch available from Apple Inc. of Cupertino, Calif.).

In addition, patients are able to save various treatments, trials, and news, and take notes and log their treatment journey. Finally, in at least one embodiment, the system can provide automated notifications to prompt the user when information is updated or to enter new data and take surveys.

The method begins 201. As a first step, software (such as patient mobile app 110) is installed 202, and user account information is collected 203. In at least one embodiment, the user can enter user account information manually; alternatively, such information can be retrieved from any suitable location, such as from patient identifying information 120 and/or from other sources.

In at least one embodiment, prior to such direct retrieval of patient data, the user is given the option 204 of authorizing patient record access or answering medical questions. If the user chooses to authorize patient record access, he or she can sign an electronic consent directly within patient mobile app 110. App 110 then automatically generates the consent form in a format that is acceptable to the third party that has whatever record(s) is/are being requested; this third party may be, for example, a medical hospital or a diagnostic lab. This may include collecting 205 user-identifying information and/or HIPAA record authorization.

The system then automatically sends the request to the third party, along with any other information that may be needed to retrieve the desired information. The request can be sent by any suitable means, such as for example via email, fax, or API integration. This may include transmitting 206 encrypted user-identifying information and record authorization to a server. Once the third party responds with the requested information, data records can be automatically processed and analyzed by the system to extract the desired information, according to the steps described below.

Examples of patient data include but are not limited to electronic medical records, genomic information, claims records, geolocation, diagnostic tests, medical benefits, insurance plan information, and the like. Examples of health care systems that can be the source of the requested information include but are not limited to hospital systems, physician clinics, payers systems, diagnostic companies, clinical labs, and the like.

If, in step 204, the user indicates that he or she would like to answer medical questions instead of authorizing access, the system collects 207 user-identifying information and answers to medical questions, for example by prompting the user via patient mobile app 110 and receiving input from the user. The collected information is then encrypted and transmitted 208 to a server for secure storage.

The system them divides 209 the collected and/or retrieved information into patient-identifying information and de-identified information. These can be stored in two separate data storage devices, such as data storage 120 for patient-identifying information and data storage 121 for de-identified patient health information. In at least one embodiment, information is de-identified by automatically searching for patient identifiers, and replacing them with code names.

In step 110, the system determines whether the user has authorized patient record access. If so, patient records are retrieved 211 and then processed 212 to extract answers to medical questions.

The system then supplements 213 any existing medical answers with new medical question answers, such as those collected in step 207.

Retrieved and/or collected patient information is then used as input into a personalization algorithm that calculates specific treatment options for the patient. In at least one embodiment, this is performed by reducing 214 the set of all possible treatments and treatment paths to those recommended for patients who match the user's medical question answers or diagnostics information.

The system then provides 215 all treatment path permutations and associated content to the user. This may include displaying treatment options, recommendations, and/or other information via patient mobile app 110. In at least one embodiment, treatment options are displayed in ordinary language to facilitate proper understanding. In at least one embodiment, the translation of treatment options to ordinary language can be performed automatically. This can be done, for example, by looking up the various options in a patient-friendly translation dictionary whereby every medical concept is translated into patient-friendly simplified language. Treatment options include but are not limited to: approved drugs, interventions such as surgery or radiation therapy, and clinical trials.

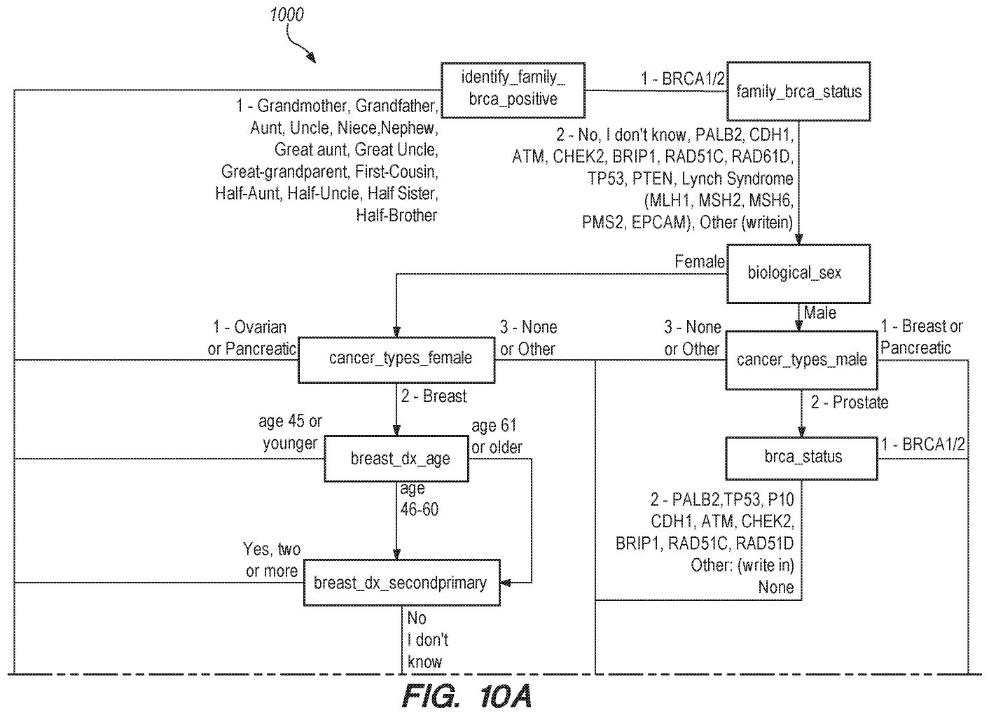

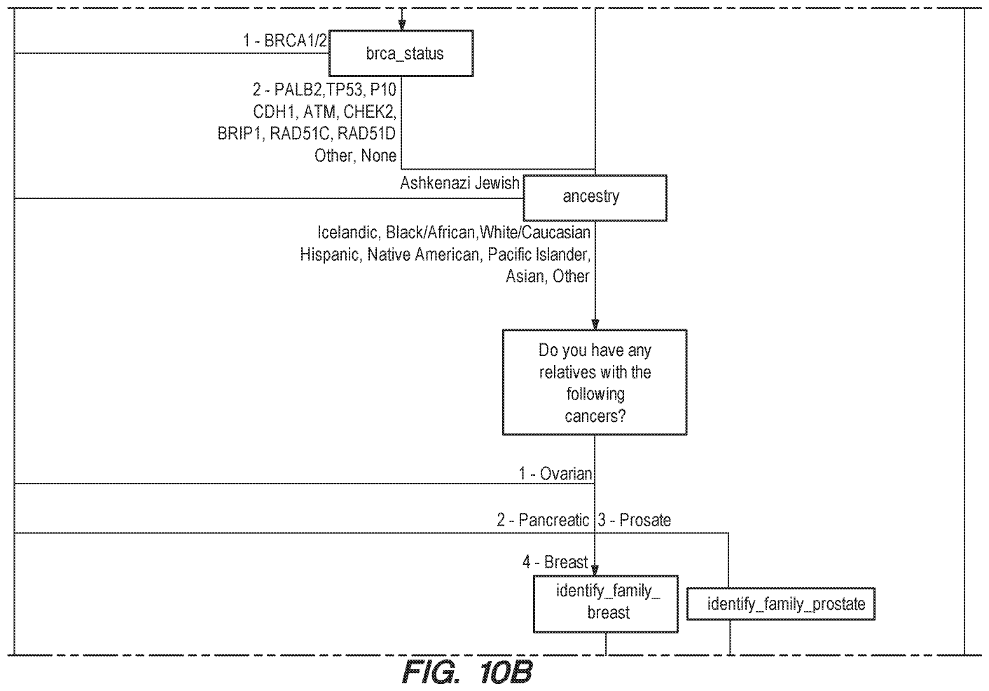

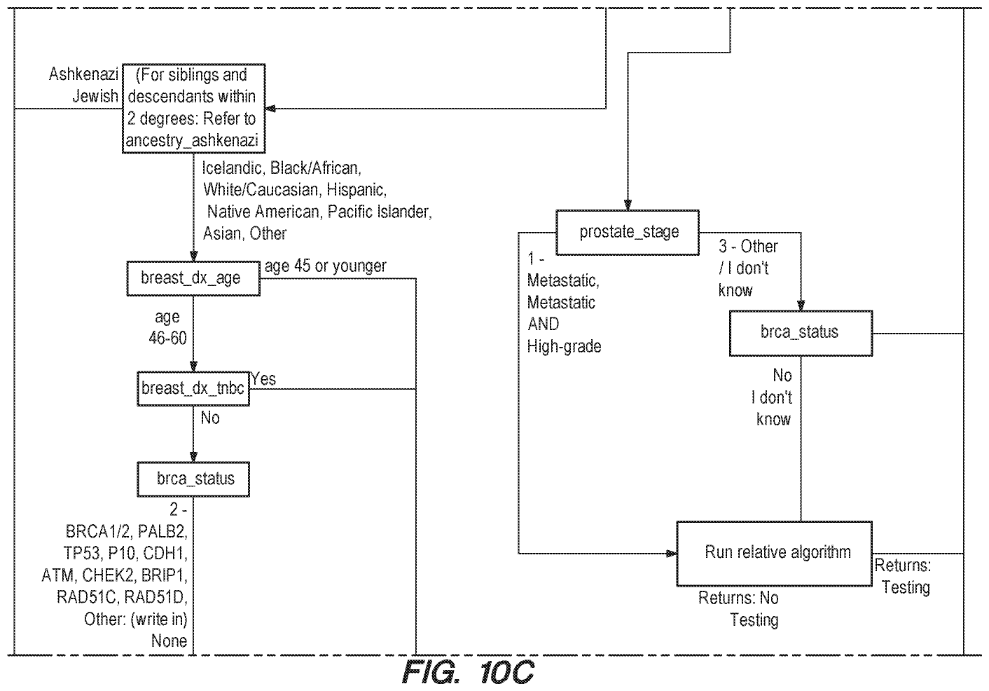

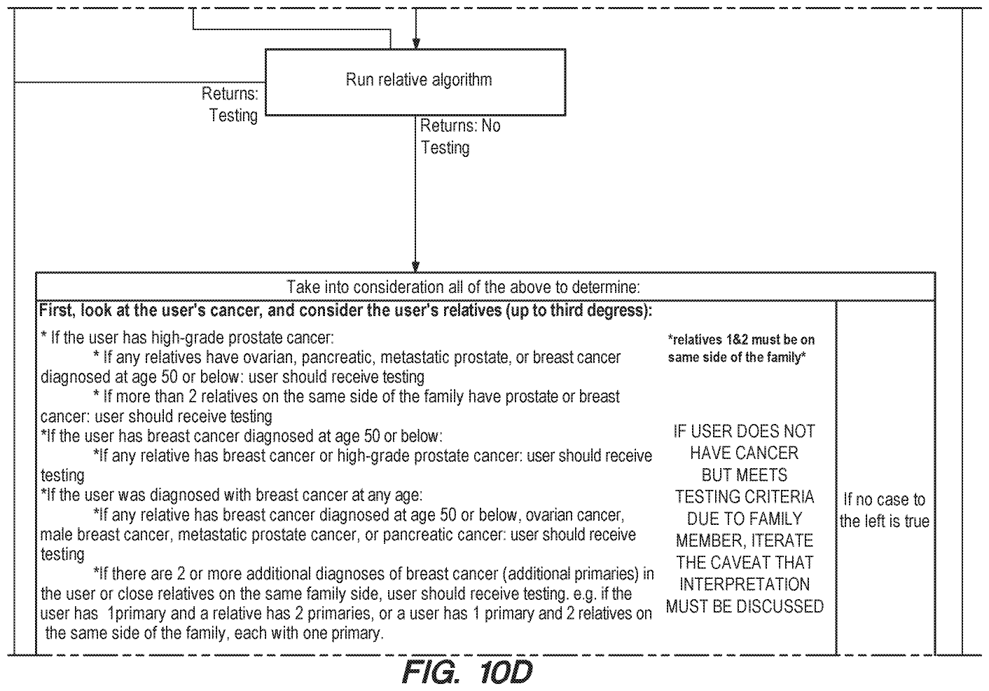

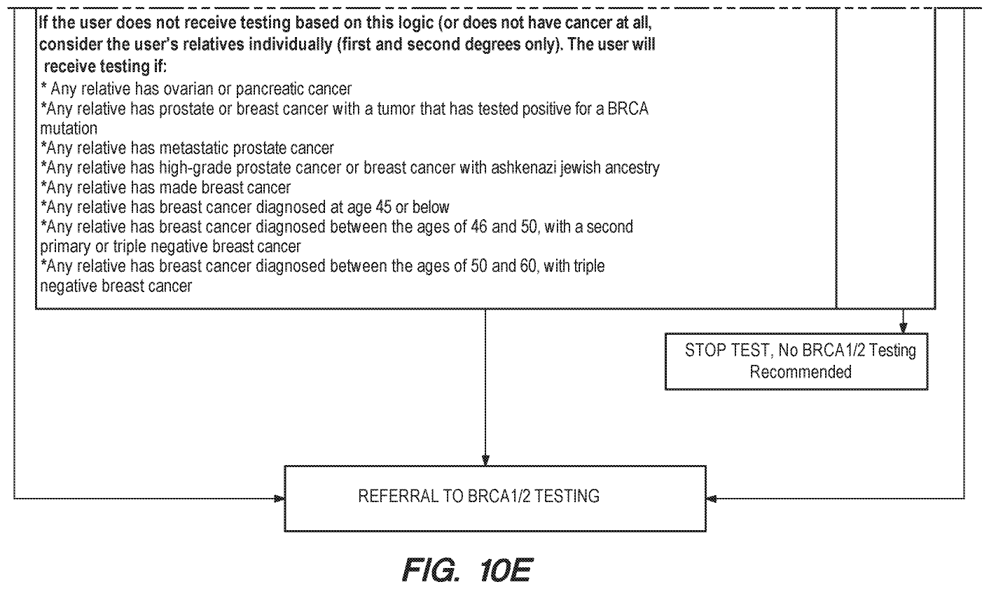

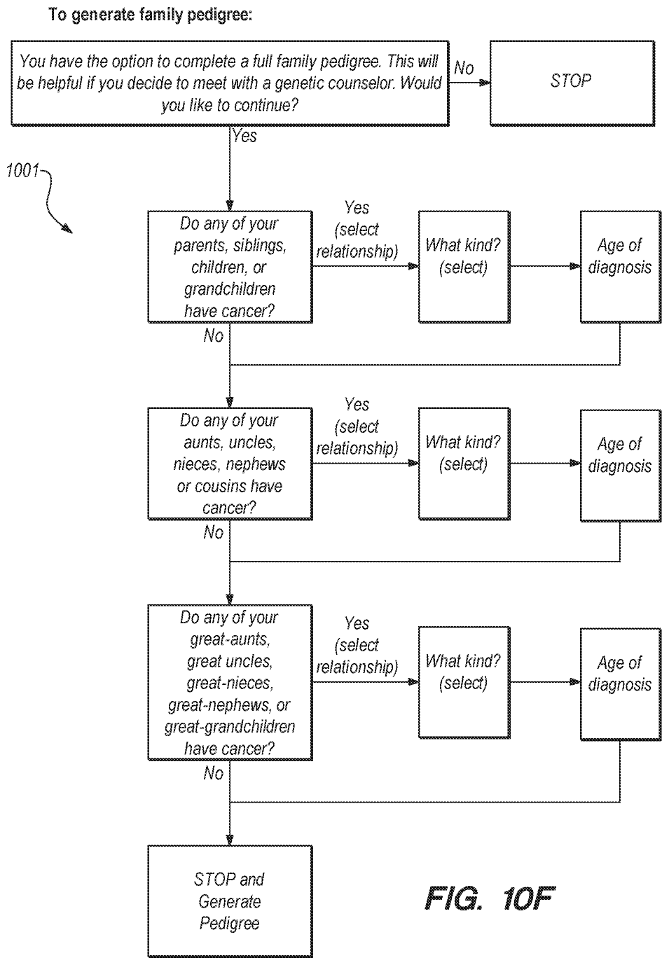

Referring now to FIGS. 10A through 10F, there is shown an example of an algorithm 1000 for personalized screening according to one embodiment, including an optional questionnaire 1001 for collecting family pedigree.

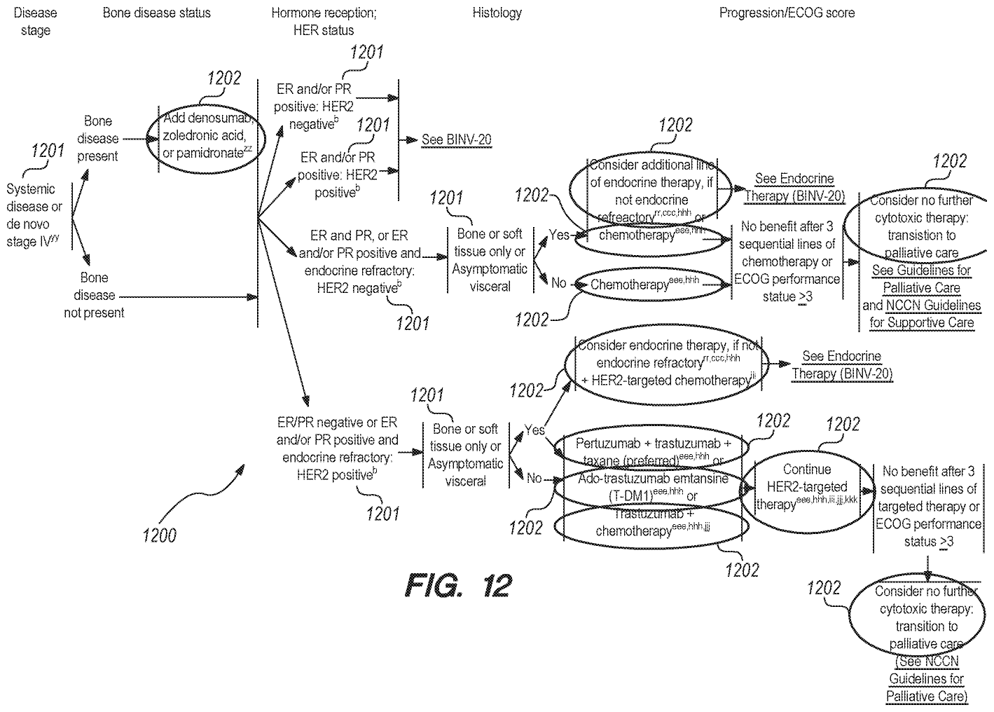

Referring now to FIG. 12, there is shown an example 1200 of an application of a personalization engine to generate appropriate treatment options for a patient, according to one embodiment. Example 1200 depicts a treatment “tree” coded with treatment options corresponding to codified objects. In at least one embodiment, these treatment options are referenced to build a treatment database, which can be stored in data storage 119 or in some other suitable location. As shown in FIG. 12, the treatment tree includes diagnostic questions 1201 that lead to various treatment options 1202.

In at least one embodiment, the underlying decision tree for the personalization engine is adapted from existing health care guidelines. In at least one embodiment, these existing guidelines are adapted and transformed into one or more algorithms, with questions, answers, and options; the algorithms are then used by the personalization engine to generate recommendations.

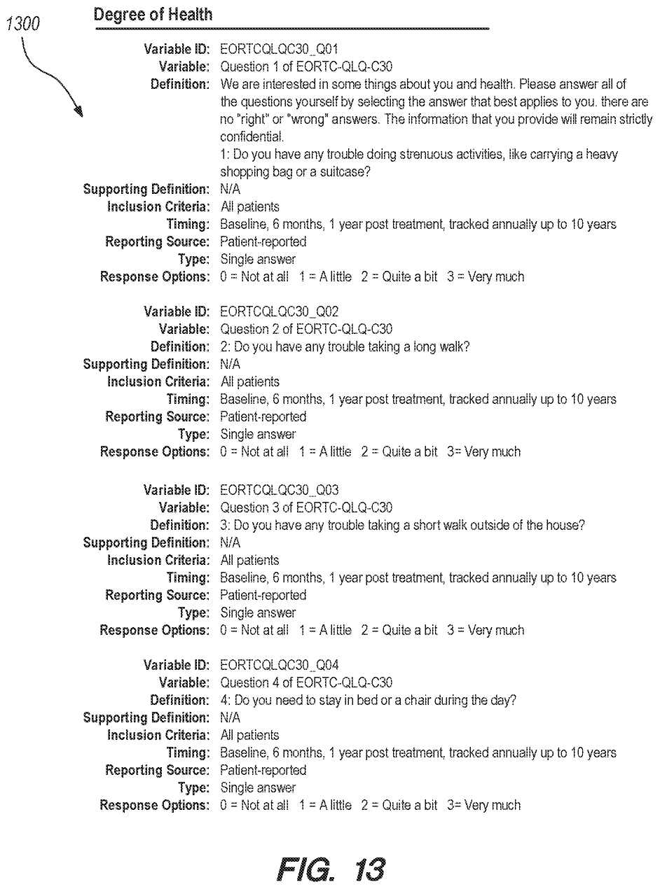

Referring now to FIG. 13, there is shown an example 1300 of input received from a patient-reported outcome (PRO) service, according to one embodiment. In at least one embodiment, such input can be provided from a third-party PRO service, collected via a questionnaire or by other means. This input can be automatically retrieved via data API 116 or by accessing database 120, and displayed on patient app 114 to collect the patient data. The resulting output can then be incorporated into the described system, for purposes of providing feedback and/or improved recommendations, particularly when integrated with clinical data, and other third party healthcare data.

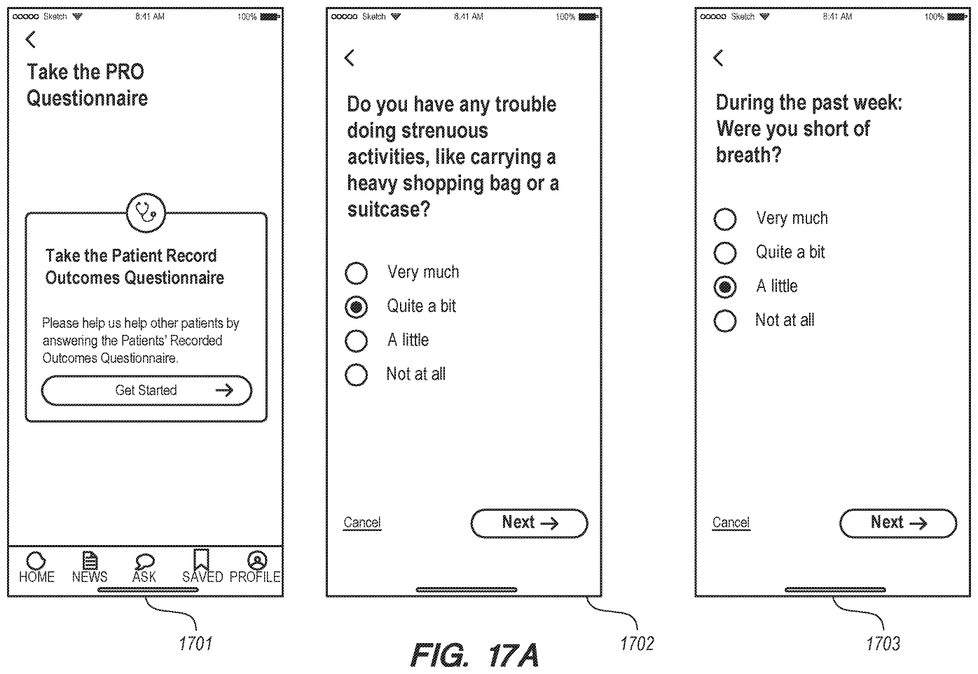

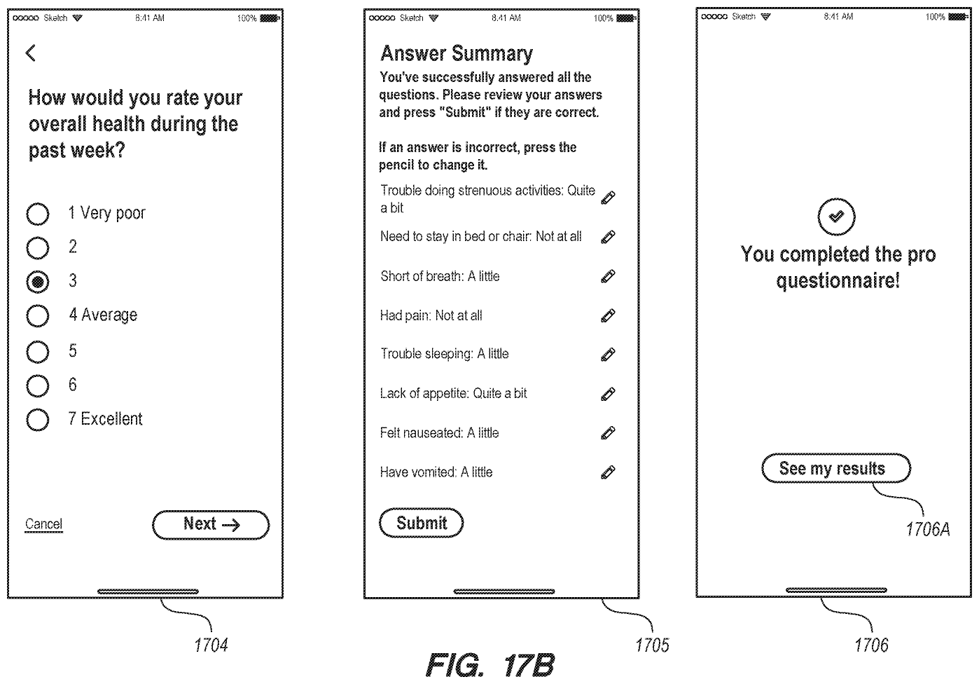

Referring now to FIGS. 17A and 17B, there are shown various examples of screens of a user interface for completing a Patient Record Outcomes Questionnaire, according to one embodiment. Screen 1701 provides an introduction. Screens 1702, 1703, and 1704 prompt the patient for information to be collected. Screen 1705 displays an answer summary. Screen 1706 displays confirmation that the questionnaire has been completed, and provides a link 1706A to results.

In at least one embodiment, the information provided by the user or patient in response to the questionnaire can be used to match the patient with other patients that have similar symptoms, patient reported outcomes, and/or issues. In at least one embodiment, the system can automatically and anonymously connect patients with other patients, based on similarity of symptoms, patient reported outcomes, and/or other factors. Such connection can take place via any suitable mechanism, including by text, email, social media, and/or the like. In at least one embodiment, the patient's demographics, preferences, and/or other factors may also be taken into account when suggesting matching patients for such communications and connections.

### User Experience

The system described herein employs a proprietary personalization engine to implement an integrated mobile platform that brings together all the information needed to manage a patient's care in one place. Patients can use mobile app 110 to readily access expert-validated, evidence-based personalized treatment information, gain access to their health records, find relevant clinical trials available in their area, and input symptoms and quality of life data, and be matched with similar patients based on their clinical histories and personal preferences.

The described system thus provides an integrated mobile platform for direct use by patients and patient navigators, as well as a shared provider module, creating a comprehensive longitudinal data platform. The system can also be extended to include survivorship care navigation so as to deliver a complete view of patients' personal clinical information as well as up-to-date, personalized, clinically-validated care planning and navigation. The system and its platform thus provide mechanisms to empower patients to have a greater, more educated role in their own care, by facilitating informed decision-making, physician-patient communication, and care management.

The integrated methodology provided by the system described herein enables all individuals to make informed decisions about their medical care, while allowing scarce navigators and provider resources to be utilized efficiently. Specifically, in various embodiments, the described system can include any or all of the following advantages over prior systems:
Integration of evidence-based clinical guidelines with a novel direct-to-patient mobile experience: The system integrates the NCCN clinical guidelines in a patient-friendly language and format, making them broadly accessible and avoiding the need for provider parsing and translation of treatment options
Seamless creation and integration of a Secure Care Plan (SCP): The system can provide an EHR-integrated SCP that enables providers across disconnected healthcare systems to effectively manage care of patients.
Automation of screening tools: The system can integrate provider-driven and validated risk-screening tools so as to free up precious provider resources in the risk-detection setting and streamline risk detection.
Personalized navigation: The system offers a personalized geo-enabled approach that helps individuals gain access to local resources, facilitating navigation, especially in low-resource settings. This allows patients to be connected to the clinical trials that are closest to them, as well as to local resources. In at least one embodiment, integrated GPS functionality of the patient's phone (or other device) is used to identify the patient's location. Based on this information, the patient is presented with resources specific to his or her geographic location and based on his or her specific health history and needs, including for example patient assistance resources.
Dynamic live updates: In at least one embodiment, the system uses adaptive algorithms to provide automatic push updates so that both users and providers can take informed action. This includes updates to guidelines, latest scientific findings, and/or the like.

### User Interface Example

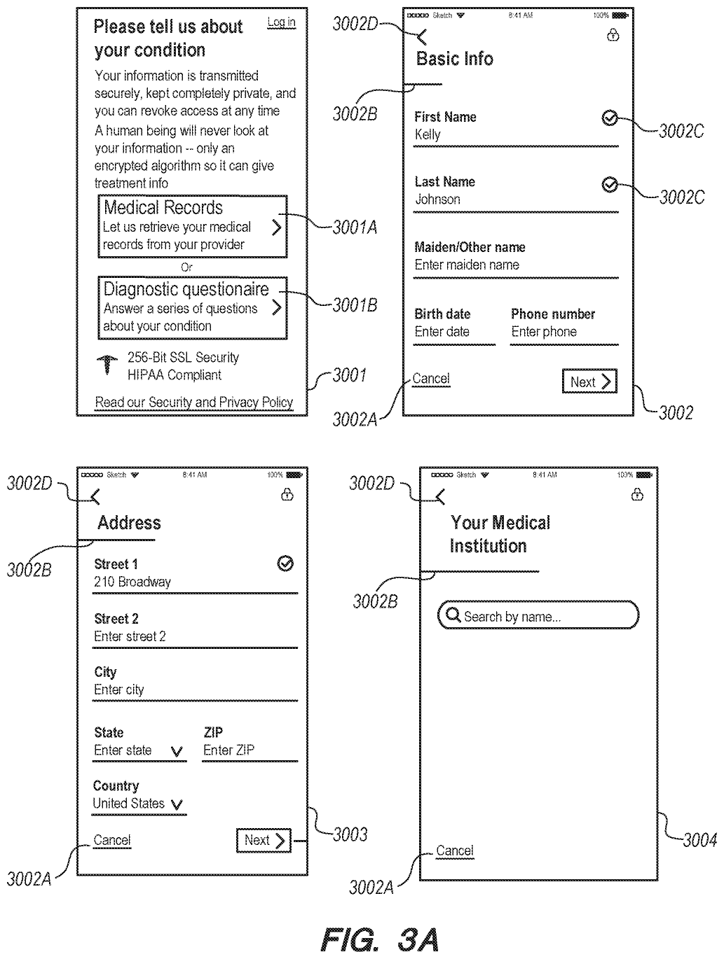

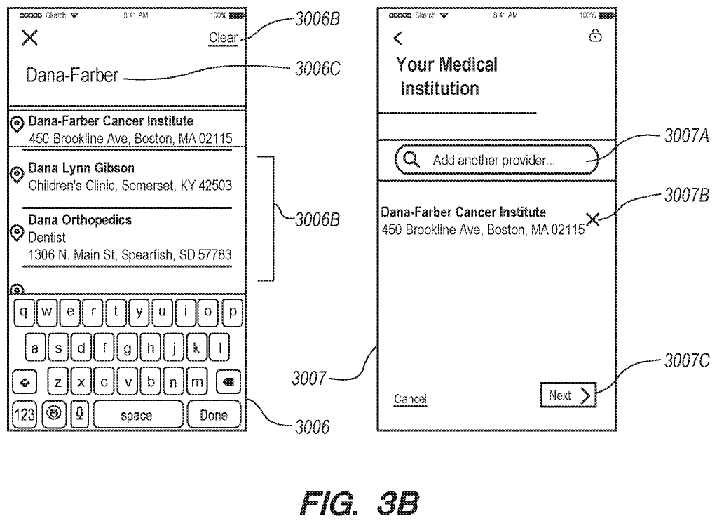

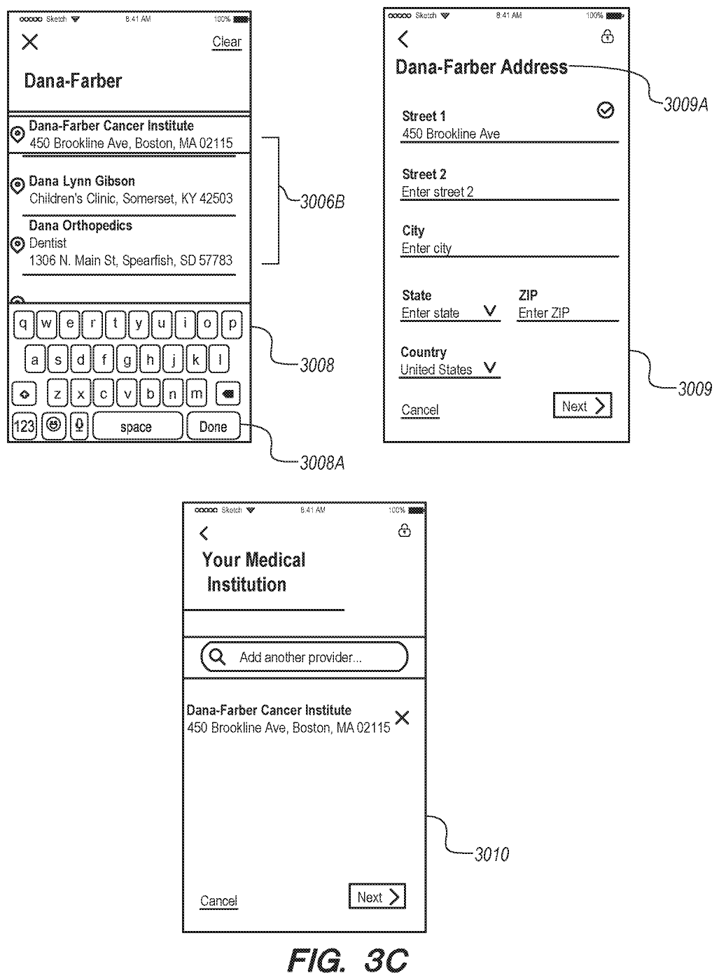

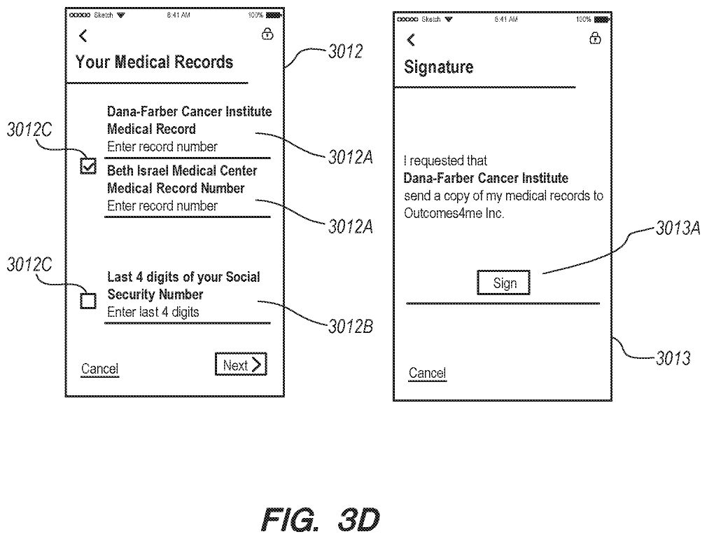

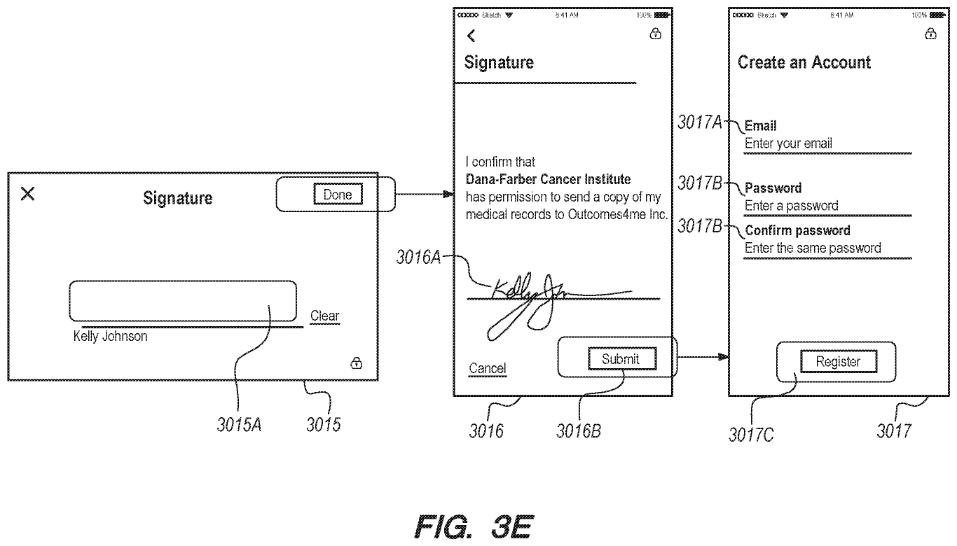

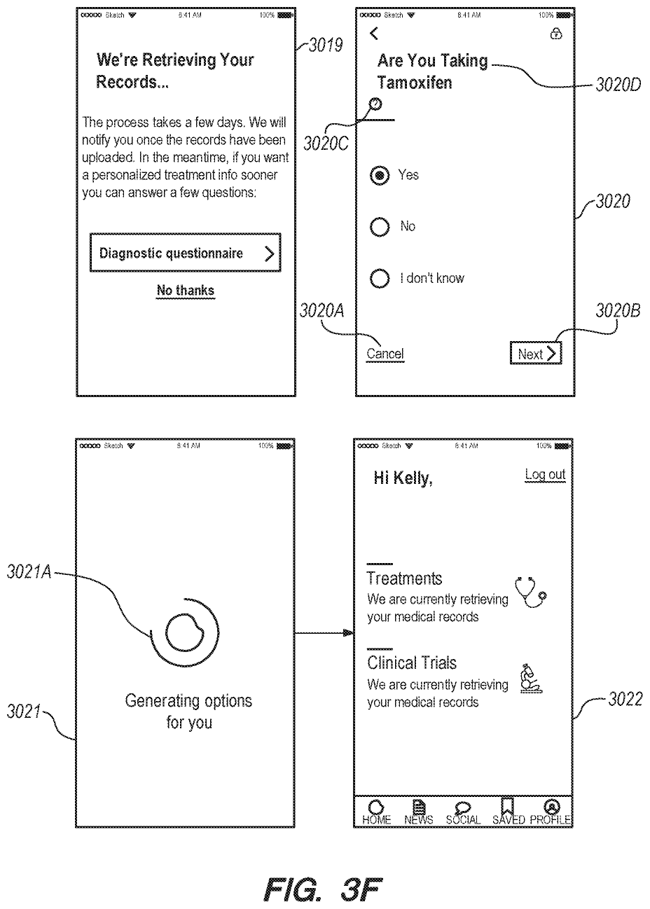

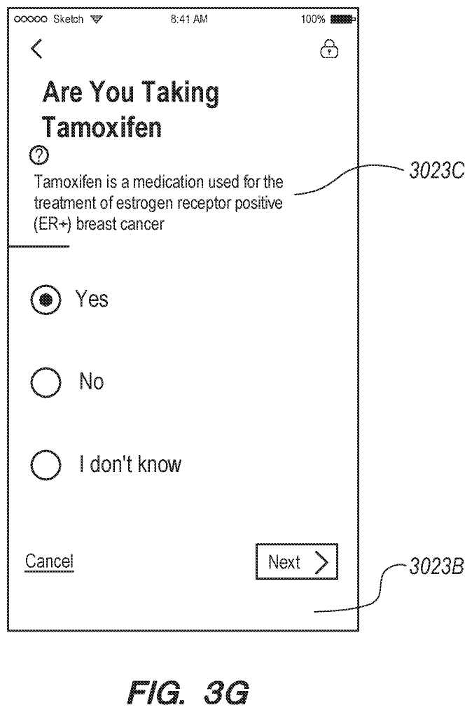

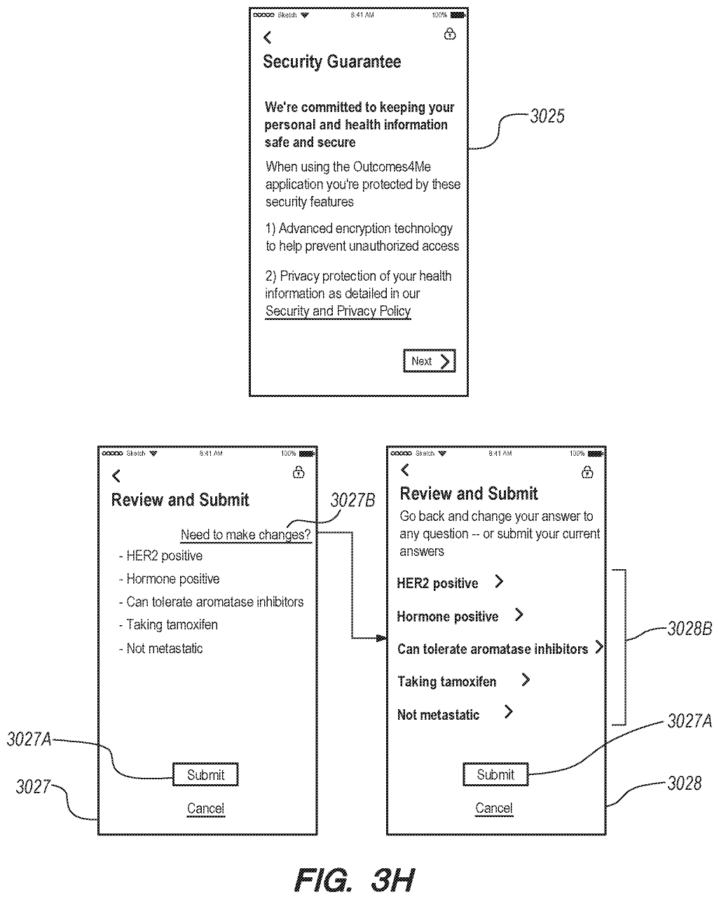

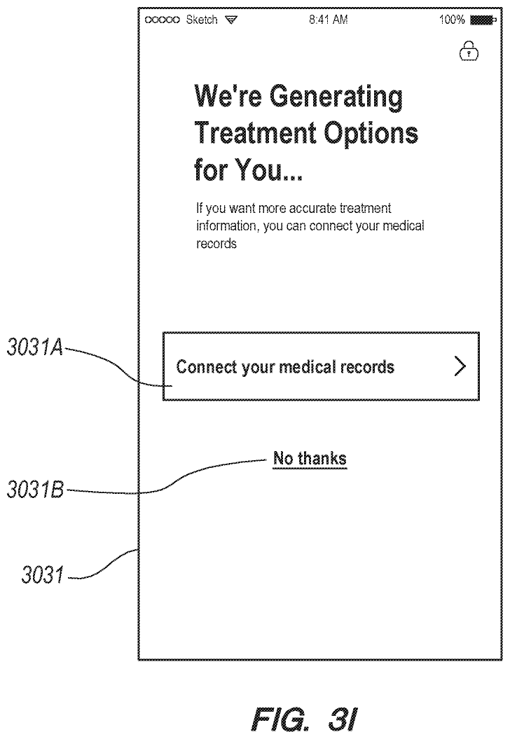

Referring now to FIGS. 3A to 3I, there are shown various examples of screens of a user interface for medical records workflow according to one embodiment.

In FIG. 3A, the user enters information to allow access to the patient's medical records. In screen 3001, the user is prompted to either allow access to his or her medical records by tapping on button 3001A, or answer a series of questions about his or her condition by tapping on button 3001B.

In screen 3002, the user has selected the option to allow access to his or her medical records, and is prompted to input basic information. In at least one embodiment, a cancel button 3002A is provided on screen 3002 and subsequent screens. Clicking on cancel button 3002A returns the user to initial request screen 3001. This continues until the user has finished the medical records flow and reached the home page. In at least one embodiment, progress bar 3002B indicates the user's progress in any multi-screen processes. In at least one embodiment, in screen 3002 and subsequent screens having more than one form input, checkmark 3002C is displayed after the user successfully completes each input.

In at least one embodiment, back button 3002D is provided on screen 3002 and subsequent screens. Clicking on back button 3002D returns the user to the previous screen. This continues until the user has finished the medical records flow and reached the home page.

In screen 3003, the user is prompted to input his or her address. In screen 3004, the user is prompted to initiate a search for his or her medical institution.

FIG. 3B depicts further details of the process for allowing access to medical records by selecting a medical institution. Again, in screen 3004, the user is prompted to initiate a search for his or her medical institution. In screen 3006, results of the search are shown, and the user can tap on a search result to select it. In at least one embodiment, search results 3006B are displayed as the user types input in field 3006A. Clear button 3006C clears the search input from field 3006A.

Screen 3007 shows the selected medical institution. The user can confirm the selection by tapping on Next button 3007C, or remove the selection by tapping X 3007B, or initiate another search by tapping in field 3007A.

FIG. 3C depicts further details of the process for allowing access to medical records by adding a new medical institution. In screen 3008, results 3006B of the search are shown, but the desired result is not shown; the user can tap “Done” button 3008A to add a new medical institution. In screen 3009, the user can enter information about the new medical institution. Title 3009A for the new medical institution is taken from the text the user previously entered in search input field 3006A of screen 3006. Screen 3010 prompts the user to confirm the new medical institution.

FIG. 3D depicts further details of the process for allowing access to medical records. In at least one embodiment, the user accesses screen 3012 by tapping on Next button 3007C in screen 3007. Screen 3012 prompts the user to enter record number(s) in field(s) 3012A and/or his or her social security number in field 3012B. In at least one embodiment, if the user has completed inputs in both fields 3012A and 3012B, he or she can clarify his or her choice by tapping on one of checkboxes 3012C. In screen 3013, the user is prompted to sign the form, by tapping on Sign button 3013A, to allow access.

FIG. 3E depicts further details of the process for allowing access to medical records. Again, in screen 3013, the user is prompted to sign the form, by tapping on Sign button 3013A, to allow access. Screen 3015 depicts a signature area 3015A where a user can use a finger to sign. Screen 3016 prompts the user to confirm signature 3016A by tapping on Submit button 3016B. Screen 3017 prompts the user to create an account by entering an email address in field 3017A and password in fields 3017B, and tapping on Register button 3017C.

FIG. 3F depicts further details of the process for creating an account and presenting a diagnostic questionnaire. Screen 3017 prompts the user to create an account by entering an email address in field 3017A and password in fields 3017B, and tapping on Register button 3017C. In screen 3019, the user is given the option to take a diagnostic questionnaire. Screen 3020 depicts an example of a screen from such a questionnaire, wherein the user is prompted to answer a question 3020D. Other questions can be presented using a similar layout or other layout. Question mark icon 3020C provides access to a dropdown element with additional information, as described below. Cancel button 3020A cancels the questionnaire. Next button 3020B causes the display to proceed to the next question.

In at least one embodiment, once the user has answered a number of questions, the system generates treatment options for the patient; screen 3021 depicts an example of a loading screen that may be shown while the back-end processes of the system generate treatment options. In at least one embodiment, a circular progress bar 3021A is shown to indicate progress as the back-end system generates treatment options. Screen 3022 is an example of a home screen indicating that treatments and/or clinical trials are being retrieved.

FIG. 3G depicts an example of a user interaction with a diagnostic questionnaire. In screen 3023B, in response to the user tapping on question mark 3020C of screen 3020, additional information about the drug referenced in the question is shown in a dropdown element 3023C.

FIG. 3H depicts additional examples of a diagnostic questionnaire flow. In screen 3025, the user is assured that his or her personal and health information will be kept safe and secure. Screen 3027 depicts a questionnaire review. The user can click on Submit button 3027A to submit his or her responses, or can click on button 3027B to make changes. Clicking on button 3027B takes the user to screen 3028, where the user can click on any button 3028B to change a previously entered result, and/or can click on Submit button 3027A to submit his or her responses. In at least one embodiment, the user can click on any of the responses in screen 3028 to return to that question in the questionnaire flow. The user then continues to retake the subsequent questions in the questionnaire. In at least one embodiment, while retaking the questionnaire, if the user encounters any questions that have already been answered, these questions are automatically skipped and not presented to the user; his or her previous answer remains. Thus, in at least one embodiment, the user is only presented with questions that he or she has not previously answered.

If user taps on Submit button 3027A in screen 3027, and has not yet created an account and connected his or her medical records, screen 3017 is presented, allowing the user to register, as described above in connection with FIG. 3E. Referring now to FIG. 3I, screen 3031 is depicted, giving the user an opportunity to connect his or her medical records. The user can accept by tapping on button 3031A, or decline by tapping on button 3031B. If the user accepts, he or she is taken to screen 3002 as described above in connection with FIG. 3A. If the user declines, he or she is taken to screen 3021, depicting an example of a loading screen that may be shown while the back-end processes of the system generate treatment options, as described above in connection with FIG. 3F. The user can then be taken to screen 3022, as described above in connection with FIG. 3F.

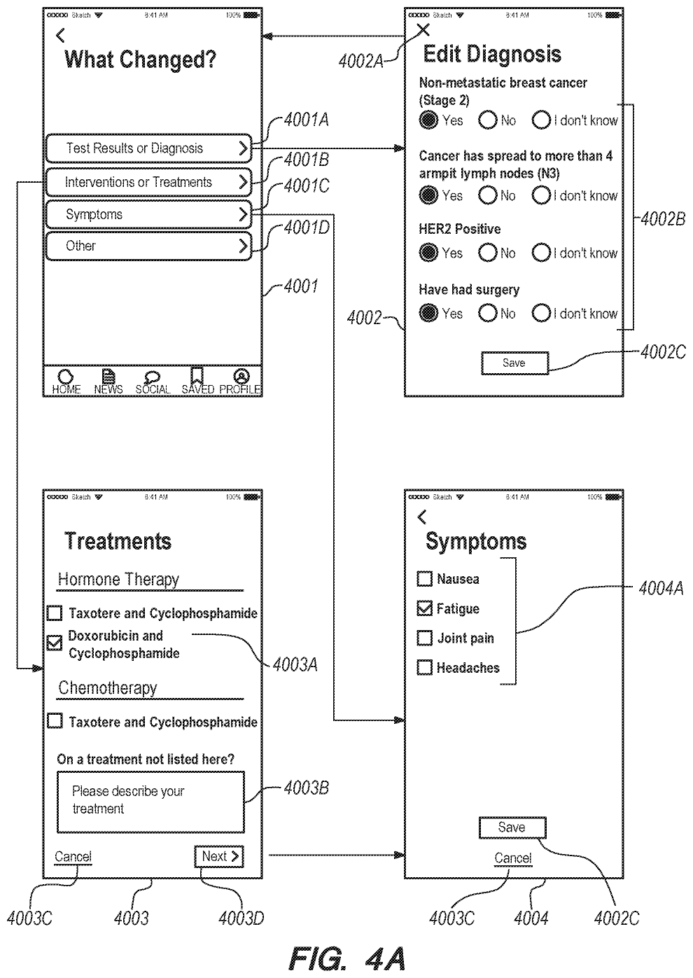

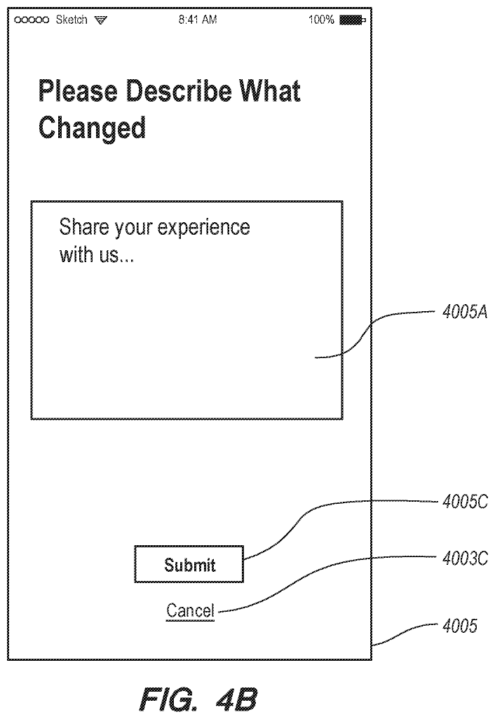

Referring now to FIGS. 4A and 4B, there are shown various examples of screens of a diagnosis update according to one embodiment. In screen 4001, the user can select from a number of buttons 4001A through 4001D to provide additional or changed information, including test results, diagnosis, interventions, treatments, and symptoms. The various buttons 4001A through 4001D take the user to different screens to display and/or change information. A button 4001D labeled “other” provides access to screens for automatic and direct personalization of information.

In screen 4002, the user has tapped on Test Results or Diagnosis button 4001A. The user can update answers to various questions 4002B, and can click on Save button 4002C to return to the home page with changes saved. Clicking X button 4002A returns the user to the diagnosis update screen 4001 and cancels any changes made.

In screen 4003, the user has tapped on Interventions or Treatments button 4001B. The user can update answers to various questions 4003A, and/or can enter further description in field 4003B. In at least one embodiment, screen 4003 is preloaded with any checkbox selections previously made by the user. The user can click on Next button 4003D to proceed with screen 4004, or can click on Cancel button 4003C to return to the diagnosis update screen 4001 and cancel any changes made.

In screen 4004, the user has tapped on Symptoms button 4001C. The user can update input for any symptoms 4004C. The user can click on Save button 4002C to return to the home page with changes saved. Clicking Cancel button 4003C returns the user to the diagnosis update screen 4001 and cancels any changes made.

In screen 4005, the user has tapped on Other button 4001D. Here, user can enter free text in text box 4005A. Submit button 4005C saves the input and returns the user to the home page. Cancel button 4003C returns to the diagnosis update screen 4001 and cancels any input.

### Automated Integration of Clinical Guidelines

Many diseases have clinical treatment guidelines aimed at helping physicians identify treatment options for patients. These guidelines are often issued by provider associations; examples include the National Comprehensive Cancer Network (NCCN) or the American Society of Clinical Oncology (ASCO). Conventionally, guidelines are disseminated, for example in PDF format, for physicians to review and determine treatment options for patients.

In at least one embodiment, the system described herein takes as input patient clinical information, and outputs treatment options according to set clinical guidelines. In at least one embodiment, a treatment personalization method similar to that depicted in FIG. 2 is used to select which treatments to display.

In various embodiments, different treatment personalization algorithms may be provided for different diseases. Clinical guidelines are used as input to specify the algorithm to be used. Patient clinical information is provided as input to the system. The output is the specific treatment option according to the set guidelines.

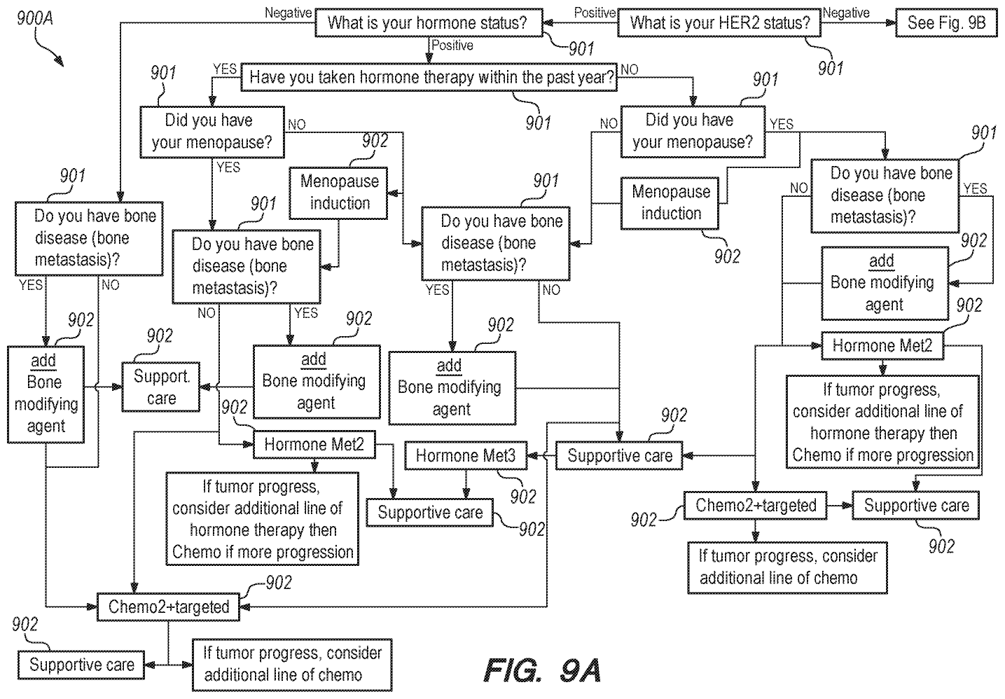

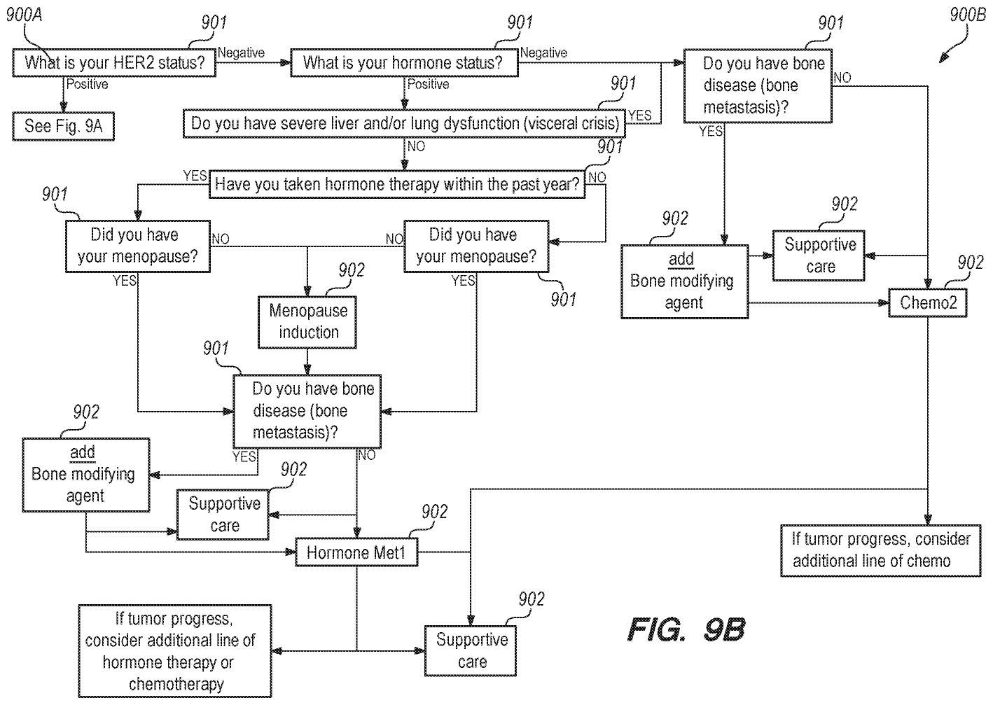

Referring now to FIGS. 9A and 9B, there is shown an example of a treatment personalization algorithm 900A, 900B for metastatic breast cancer. FIG. 9A depicts treatment personalization algorithm 900A for HER2 status positive, and FIG. 9B depicts treatment personalization algorithm 900B for HER2 status negative. Patient clinical information is expressed as answers to questions 901. In response to replies to such questions 901, treatment options 902 are provided. In at least one embodiment, an algorithm such as personalization algorithm 900A, 900B is automatically implemented by the described system, according to the method depicted in FIG. 2 or any other suitable method.

In at least one embodiment, the personalization algorithm determines the shortest path, which then maps to the smallest number of questions that the algorithm needs to know to resolve the case and provide options.

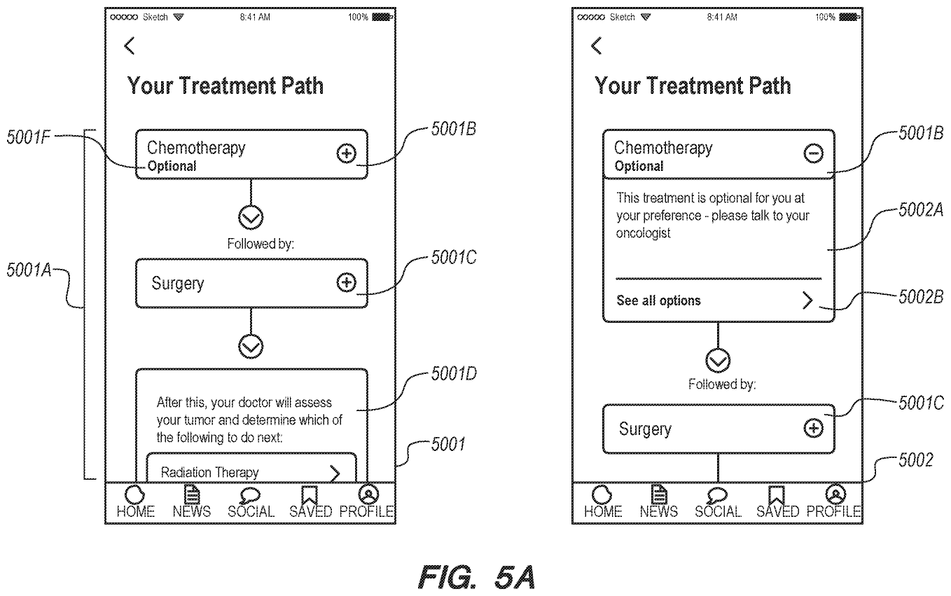

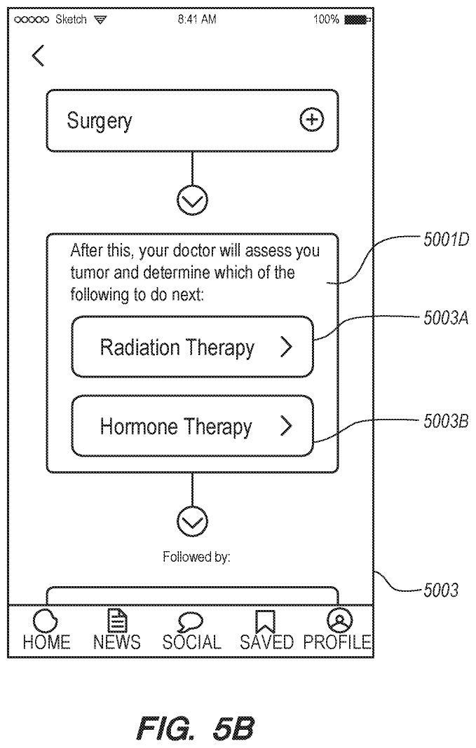

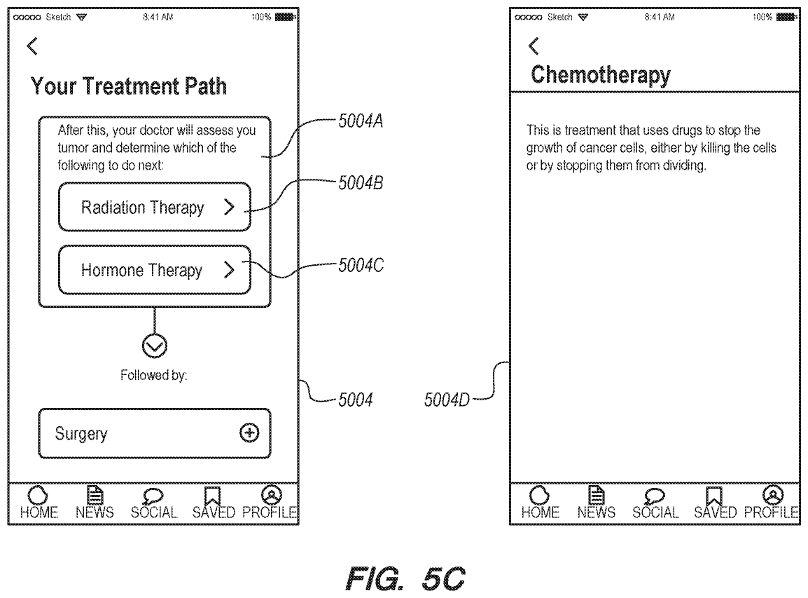

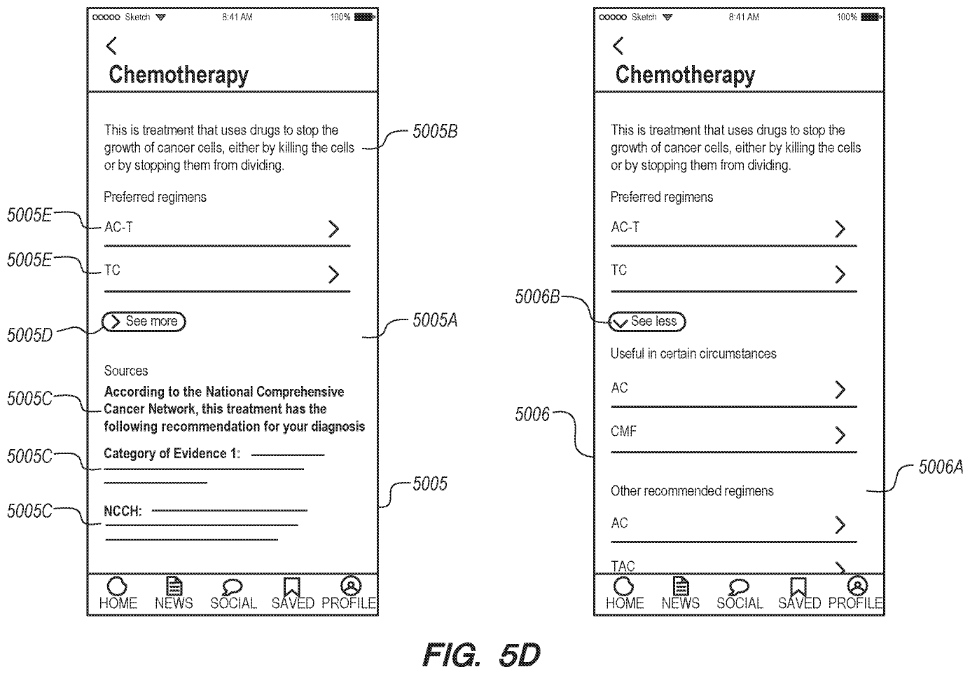

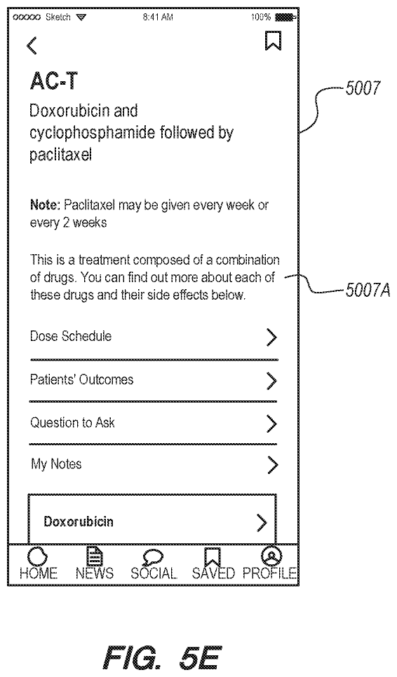

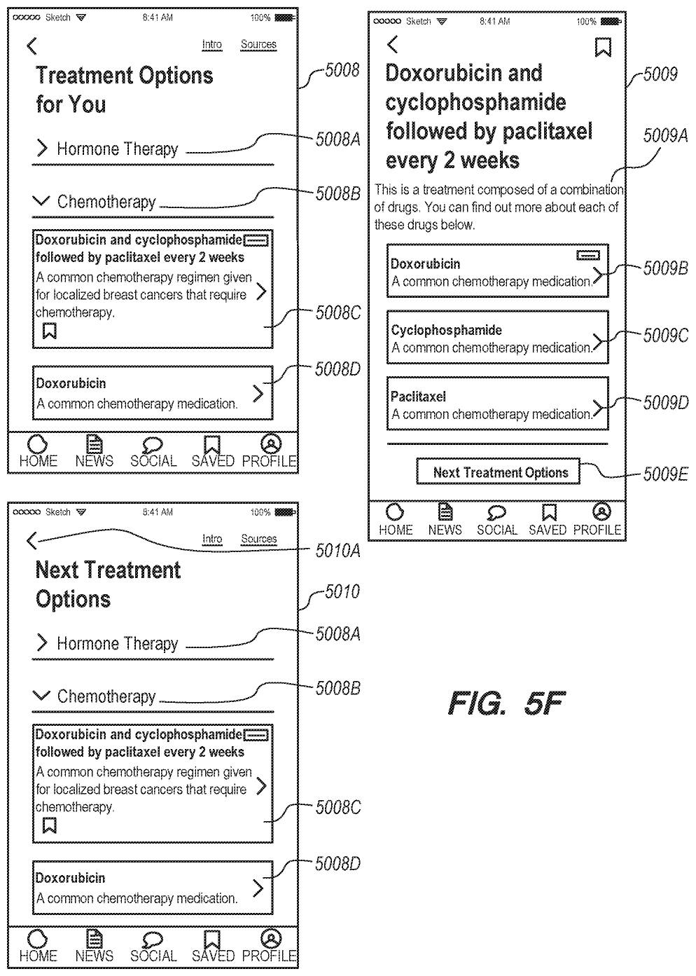

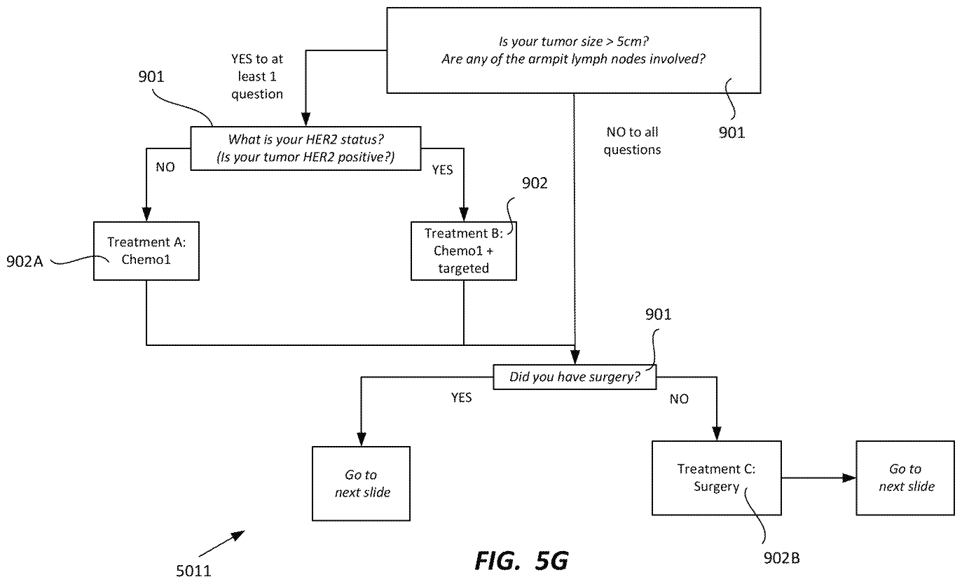

In at least one embodiment, the system presents a treatment path including various treatment categories for a patient, in a linear format. Referring now to FIGS. 5A to 5F, there are shown examples of screens of a user interface for a treatment path according to one embodiment.

FIG. 5A depicts screen 5001, in which a treatment path 5001A is shown, including various treatment steps 5001B, 5001C, and card 5001D. The user can scroll screen 5001 as needed to see additional steps that may not be initially visible. In at least one embodiment, each treatment category is depicted in a distinctive color or having some other distinctive visual attribute. A text label 5001F can indicate whether a particular treatment step is optional. In at least one embodiment, if “supportive care” is part of a patient's treatment path, it is always placed second in the path.

In at least one embodiment, the user can tap on any of treatment steps 5001B, 5001C to expand it. For example, tapping on Chemotherapy step 5001B causes screen 5002 to be displayed, including card 5002A showing additional details for that step 5001B. In this example, card 5002A explains why step 5001B is optional for this patient. The user can tap on See All Options button 5002B to see additional options concerning step 5001B, as will be discussed in more detail below in connection with FIG. 5D. Tapping on Chemotherapy step 5001E in screen 5002 collapses card 5002A and returns to screen 5001.

FIG. 5B depicts screen 5003, in which the user has scrolled the screen so that card 5001D is visible. Card 5001D depicts the situation in which a particular treatment step may not be determined until a patient has progressed in their treatment; until then, multiple alternative treatment steps may be possible. Accordingly, in this example, card 5001D depicts multiple “to be determined” steps 5003A, 5003B that are grouped together within treatment path 5001A.

FIG. 5C depicts screen 5004, in which card 5004A indicates that the first step of the patient's treatment path is a group of “to be determined” steps 5004B, 5004C. Card 5004A therefore advises the patient to talk to his or her doctor about the treatment options indicated in steps 5004B, 5004C. In at least one embodiment, tapping on one of steps 5004B, 5004C within the group activates screen 5004D, which shows summary text describing a treatment option, but omits detailed information about treatment regimens.

FIG. 5D depicts screen 5005, which may be displayed in response to user having tapped on See All Options button 5002B in screen 5002. Additional options 5005A are shown about the chemotherapy step 5001B, including various regimens 5005E. Screen 5005 may include static text elements 5005B and/or elements 5005C that include text provided by a back-end system. The user can tap on See More button 5005D to display additional treatment options 5006A, as shown in screen 5006. The user can tap on See Less button 5006B to collapse additional treatment options 5006A and return to screen 5005.

In at least one embodiment, the user can tap on one of regimens 5005E to see additional information. FIG. 5E depicts screen 5007, which includes additional information 5007A that may be displayed after the user taps on AC-T regimen 5005E in FIG. 5D. In at least one embodiment, this additional information is taken from live data from APIs such as Data API 116. The information may be reformatted automatically, so that the user always has access to the latest information, live and in a patient-friendly format.

Referring now to FIG. 5F, there are shown examples of treatment options screen 5008, treatment details screen 5009, and next treatment options screen 5010. Treatment options screen 5008 shows various treatment options 5008A, 5008B. The user can tap on treatment options 5008A, 5008B to see additional details. In this example, the user has tapped on treatment option 5008B, to see more details 5008C, 5008D describing the chemotherapy option.

The user can then tap on details 5008C, 5008D to see additional information. Treatment details screen 5009 is an example of a screen that may be shown when the user taps on details 5008C. Additional information 5009A is displayed, along with drug descriptions 5009B, 5009C, 5009D. The user can tap on any of drug descriptions 5009B, 5009C, 5009D to see more information about a particular drug. The user can tap on Next Treatment Options button 5009E to activate next treatment options screen 5010, which contains information similar to that shown in screen 5008. Screen 5010 also includes back button 5010A, which causes the display to return to treatment page 5009.

In at least one embodiment, this additional information is taken from live data from APIs such as Data API 116. The information may be reformatted automatically, so that the user always has access to the latest information, live and in a patient-friendly format.

Referring now to FIG. 5G, there is shown a portion of a treatment personalization algorithm 5011 that can be used to populate treatment options screens such as screens 5008 and 5010, according to one embodiment. As in algorithm 900A, 900B depicted and described above in connection with FIGS. 9A and 9B, patient clinical information is expressed as answers to questions 901. In response to replies to such questions 901, treatment options 902 are provided. In this example, treatment option 902A of algorithm 5011 causes screen 5008 to be populated by information collected using relevant answers received from the user. Similarly, treatment option 902B of algorithm 5011 causes screen 5009 to be populated by information collected using relevant answers received from the user.

### Geolocation of Clinical Trials

Clinical trials are often conducted at multiple locations, with various centers administering the trial. Existing systems fail to provide any functionality for automatically matching trials based on clinical data retrieved from a health care system along with location of the user. In addition, existing systems do not provide any way to display a map of those trials that match a user's disease and that are closest to the location of the patient across various clinical trials and various healthcare systems.

Various embodiments of the described system address these deficiencies. In at least one embodiment, a computer system automatically matches and prioritizes clinical trials based on a user's clinical information and location simultaneously; the resulting information is then displayed on a map. Trials are prioritized based on both clinical information and location; unique trials are shown on an interactive map or displayed as a list.

In at least one embodiment, a treatment method similar to that depicted in FIG. 2 is used to select which trials to display. The system looks up clinical attributes from patient records and/or answers to questions, and then uses these attributes to perform a multi-layered search of clinical trials; the search can include inclusion and/or exclusion criteria. In at least one embodiment, the method uses natural language processing to determine which keywords match which part of the trials. Natural language processing can also be used to analyze medical records and health information, so that the information can be integrated with clinical trials, thereby providing improved personalization.

In at least one method, clinical trials are pre-classified based on specific disease attributes; such attributes are then automatically matched to the patients attributes to improve personalization.

In at least one embodiment, the map is automatically generated by looking up the location information from a clinical trial repository and rendering it on a maps app based on a current location of a mobile device. One example of such a clinical trial repository is clinicaltrials.gov; however, information from many different sources can be integrated, and new sources can be incorporated as desired at any time.

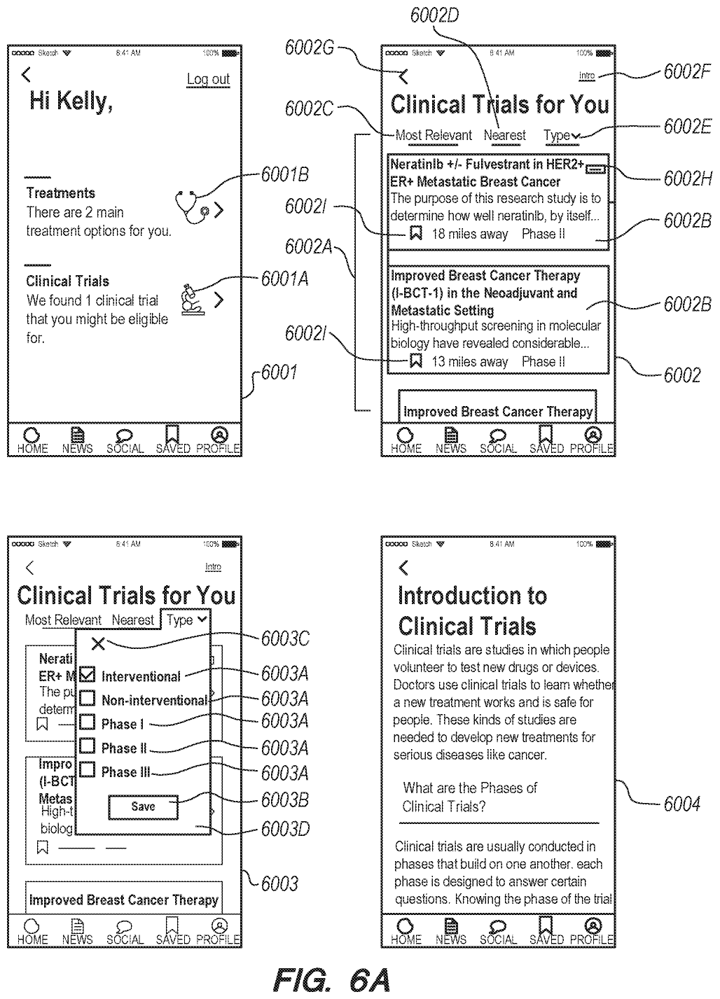

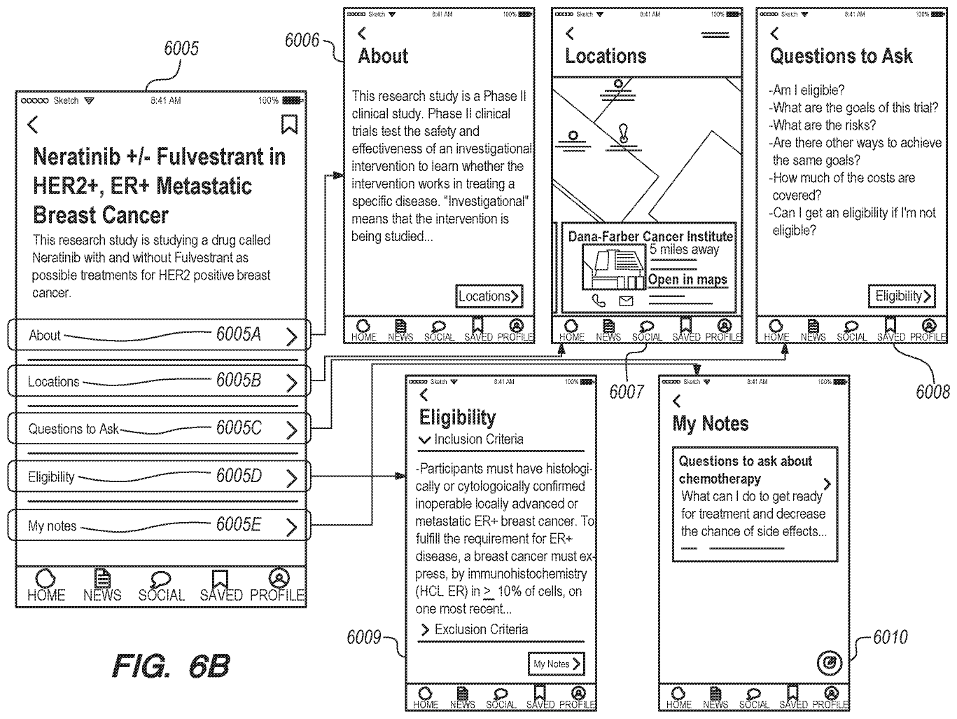

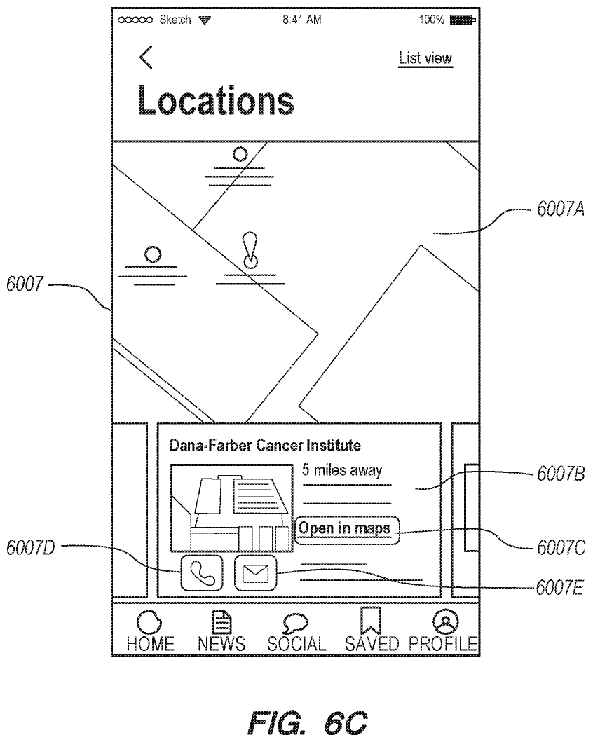

Referring now to FIGS. 6A to 6C, there are shown various examples of screens of a user interface for clinical trials workflow including GPS functionality, according to one embodiment.

FIG. 6A depicts screen 6001, in which the user is presented with options to select treatments 6001B or clinical trials 6001A. In screen 6002, the user has tapped on clinical trials 6001A, and is presented with a list 6002A of clinical trials 6002B. In at least one embodiment, GPS information is used to rank and/or filter clinical trials 6002B in list 6002A. The user can click on Most Relevant button 6002C to cause list 6002A to be ranked according to a relevancy algorithm. Nearest button 6002D causes list 6002A to be ranked based on proximity to the user's (or patient's) geographic location, as may be determined based on available GPS information. Type button 6002E causes screen 6003 to be displayed, which includes dropdown 6003D that allows the user to filter list 6002A based on type of clinical trial and/or other criteria; checkboxes 6003A are provided for such criteria. In screen 6003, Save button 6003B dismisses dropdown 6003D and saves the user's choices and returns to screen 6002. Back button 6003C dismisses dropdown 6003D and returns to screen 6002 without saving changes. In at least one embodiment, the user can also tap anywhere outside of dropdown 6003D to return to screen 6002 without saving changes.

In screen 6002, back button 6002G returns to screen 6001. Intro button 6002F causes screen 6004 to be displayed, including an introduction to clinical trials and/or other background information.

Within clinical trials list 6002A, various items of descriptive information are shown. In at least one embodiment, indicator 6002H is shown to indicate which clinical trials were added since the last time the user opened screen 6002. Distance indicator 6002I shows the distance of the clinical trial from the user's current location, based on available GPS information.

FIG. 6B depicts screen 6005, which provides information about a clinical trial. The user can tap on buttons 6005A through 6005E to see different types of information; in at least one embodiment, such information is pulled live via APIs and/or from existing stored databases. Button 6005A activates screen 6006, which provides general information about the clinical trial. Button 6005B activates map view 6007, which displays locations for the clinical trial. Button 6005C activates screen 6008, which lists questions the user may wish to ask about the clinical trial. Button 6005D activates screen 6009, which provides information on eligibility. Button 6005E activates screen 6010, where the user can add notes concerning the clinical trial.

FIG. 6C depicts further details of map view 6007, which provides an interactive map 6007A of filtered clinical trial locations. In at least one embodiment, map view 6007 can be activated by tapping on Locations button 6005B on screen 6005. Panel 6007B displays information about a clinical trial location. Open in maps button 6007C opens the user's default maps app with the clinical trial location. Phone button 6007D allows the user to call the clinical trial location using their phone. Email button 6007E opens the user's default email app with the email address of the clinical trial coordinator pre-populated, along with detailed information about the clinical trial.

In at least one embodiment, tapping on panel 6007B causes map 6007A to be centered on that clinical trial location. In at least one embodiment, the user can swipe panel 6007B left or right to view other locations; when the user swipes panel 6007B left or right, map 6007A centers on the location of the newly displayed panel.

### Outcomes Data Provision and Personalization

In at least one embodiment, the described system allows a patient to identify treatment options and prioritize such options based on one or more user-defined clinical outcome metrics.

In at least one embodiment, the system uses an algorithm that takes as input such factors as the patient's:
(1) clinical attributes (based on patient records and/or answers to questions; these can include comorbidities);
(2) age;
(3) geography; and
(4) treatment history (this can include current treatments as well as past treatments).

Based on this information, the system defines a “distance score” between various patients. Examples of methods to compute the distance score include the weighted average of individual parameters such as disease subtype, demographic characteristics, clinical history, age, and/or the like. In at least one embodiment, the weights can be determined based on patients' preferences. Alternatively, the distance score can be calculated by first identifying the scoring variables using principal components analysis and then averaging the value of each variable for each individual patient. Alternatively, other approaches can be used.

Using a distance threshold, the system retrieves outcomes data from similar patients and shows such data to the user. This information can then be used to rank order the treatments. Examples of outcomes data include, but are not limited to: clinical outcomes such as progression of disease, overall survival, etc.; side effects and toxicity, such as neutropenia, fatigue, etc.; quality of life outcomes such as ability to go to work, sexual health, effect on body mass, etc.

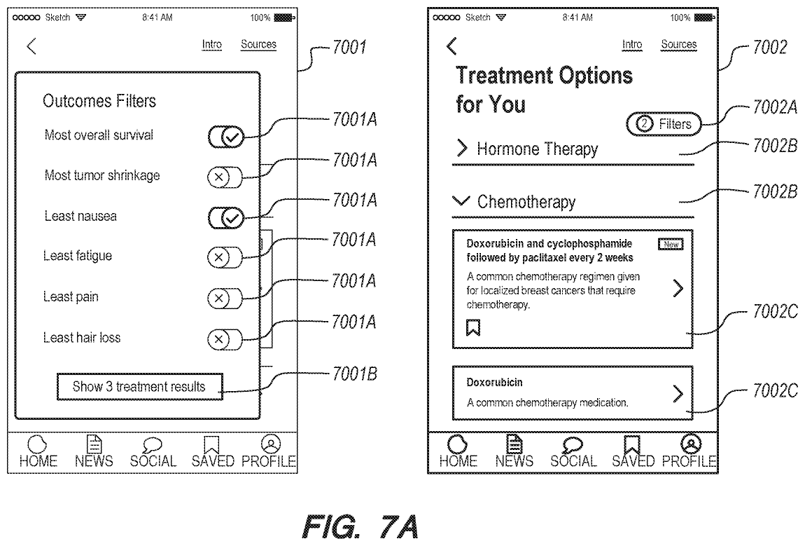

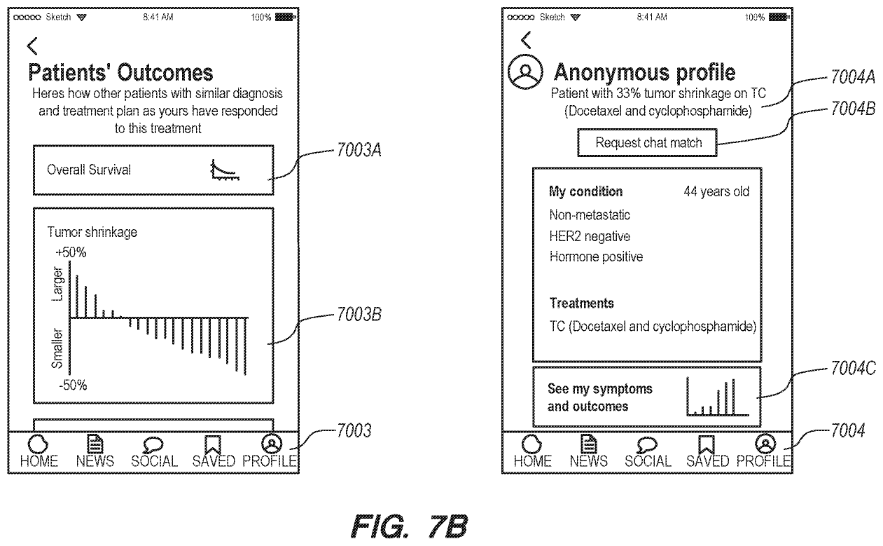

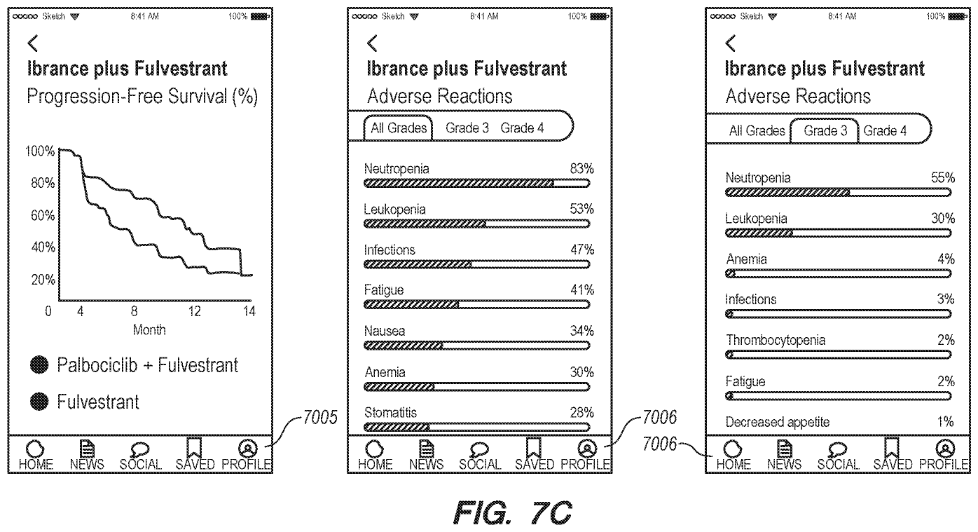

Referring now to FIG. 7A, there is shown an example of a screen 7001 of a user interface for applying outcomes filters to find treatments. The user can select among various filters 7001A to apply, and can then tap on button 7001B to see the results. Screen 7002 displays results 7002B, along with an indication 7002A of how many filters were applied. Each result 7002B can be opened to see additional information 7002C.

Referring now to FIG. 7B, there is shown an example of a screen 7003 of a user interface for presenting a visualization of patient outcomes for a particular treatment, including overall survival 7003A and bar graph 7003B depicting tumor shrinkage statistics. Each bar in graph 7003B refers to a single patient. In at least one embodiment, the user can tap on a bar in graph 7003B to activate screen 7004, which shows additional details 7004A about the patient represented by that bar. In at least one embodiment, as shown in screen 7004, a social profile can be depicted anonymously. In at least one embodiment, the user can tap on button 7004B to request to be privately matched with another patient for anonymous communication. For example, a request may be sent via notification and email to the patient in question, including anonymous information about the patient requesting the connection and their treatment journey. Button 7004C takes the user to a display of his or her own symptoms and outcomes.

Referring now to FIG. 7C, there are shown examples of screens 7005, 7006, 7007 of a user interface for presenting drug-level outcomes data, according to one embodiment.

### Determining Out-of-Pocket Costs for Treatments

Conventionally, doctors, nurses and other members of the health care team often do not know how much patients will have to pay for their care. As a result, many patients begin a course of treatment, only to be blindsided by expensive medical bills.

In at least one embodiment, the described system provides information and tools to allow for meaningful discussions of treatment cost. Specifically, tools are provided to streamline the calculation of a patient's out-of-pocket costs for treatment. Patient data is directly retrieved from health care systems and automatically analyzed. Examples of patient data include but are not limited to electronic medical records, genomic information, claims records, medical benefits and health plan structure, geolocation, designated pharmacies, diagnostic tests, and the like. Examples of health care systems include but are not limited to hospital systems, physician clinics, payer systems, diagnostic companies, and clinical labs. In at least one embodiment, the system automates the process of obtaining consent from the patient and automatically retrieving data from the provider and insurance information (including benefit spend data) from the insurer. The retrieved data is read and analyzed to determine treatment options. Treatments are then automatically provided to a cost calculator, together with treatment data and medical benefits information, to determine the out-of-pocket costs for each treatment. Treatment options can then be displayed to the patient alongside estimated out-of-pocket costs.

In at least one embodiment, the system performs cost calculation by looking up a patient's medical benefits (based on the patient's electronic authorization); this includes particulars such as co-pay amounts, and how much of their deductible has been already spent in a given year. Then, the various treatments options are analyzed and pharmacy cost is taken into account based on the patient's preferred pharmacy (such as one located in the patient's neighborhood, or used in prior orders). Out-of-pocket costs can then be automatically calculated and provided directly to patients.

In at least one embodiment, the cost data is determined on a personalized basis, taking into account information about prescribed drugs, patient benefit plan, how much of the patient's out-of-pocket maximum has been spent, and/or the like.

In at least one embodiment, treatments are automatically rank-ordered based on calculated out-of-pocket costs. In other embodiments, other factors can also be considered in performing the automatic rank-ordering.

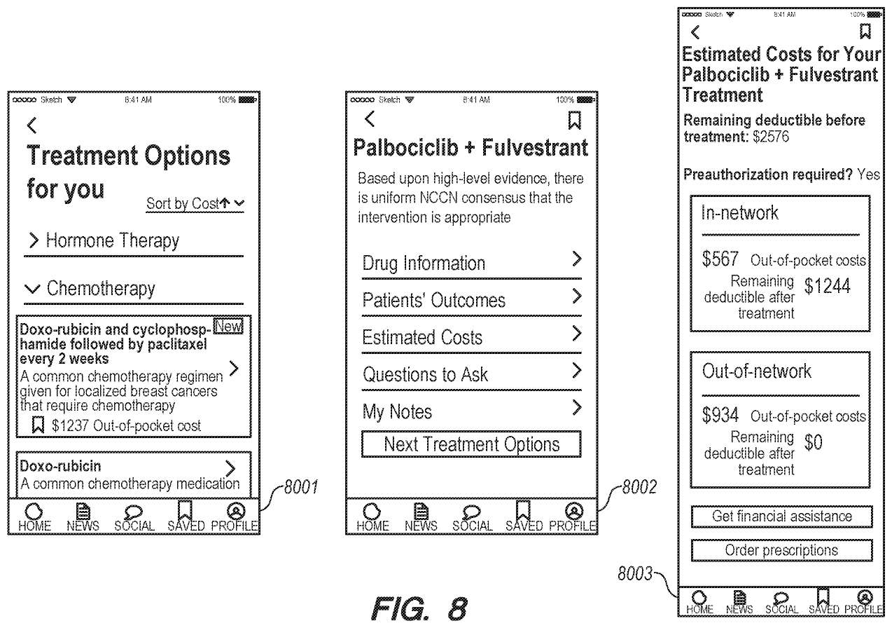

Referring now to FIG. 8, there is shown an example of a screen 8001 of a user interface for presenting treatment options, sorted by costs, and for presenting treatment details including personalized, benefit-specific patient costs. These can vary from overall costs, and can depend not only on the patient's health insurance plan, but also on how much of that plan he or she has already consumed. Screen 8002 shows details for a treatment option, including estimated costs. Screen 8003 displays additional details of the user's estimated out-of-pocket costs for the treatment option.

### Private Matching Disease-Based Social Network

In at least one embodiment, the described system provides a mechanism for patients to be automatically and privately matched with other patients that have been verified to have similar characteristics and/or diseases, and to allow such patients to interact with one another in a closed private social network. The system enforces patient anonymity.

In at least one embodiment, a social matching system is implemented based on disease attributes and personal preferences, thus allowing patients to be privately matched to other patients based on specific disease, medical history and treatment plans, social, geographic, outcomes, and/or other characteristics, including personal preferences such as age, relationship status, children's age, and/or the like. A user-initiated private social network can then be automatically created. In at least one embodiment, patient profiles are automatically verified based on patient data directly retrieved from health care systems.

In at least one embodiment, an anonymization algorithm can be provided to allow for matching to occur privately before information is revealed. This algorithm removes all patient identifiers from the patient's profile (in accordance with HIPAA requirements and/or other local health care privacy regulations), provides the patient with an ID, and displays other attributes. This allows patients to match to each other and communicate in a private encrypted setting without revealing their identities, while ensuring that the various patients' profiles have been verified based on medical records and/or other third party verified healthcare information. In another embodiment, patients can remain anonymous within the private network, so that their identifying information is never revealed.

As discussed above, the right-hand side of FIG. 7B depicts an example of a social profile for an anonymous patient, according to one embodiment.

One skilled in the art will recognize that the depicted screens, interfaces, and layouts are merely exemplary, and that the techniques described herein can be implemented using other arrangements and contexts. The depicted screens, interfaces, and layouts are presented in a form that would primarily be used by patients; however, the user interface can be adapted for use by other individuals, such as health care professionals, administrators, and/or the like.

The present system and method have been described in particular detail with respect to possible embodiments. Those skilled in the art will appreciate that the system and method may be practiced in other embodiments. First, the particular naming of the components, capitalization of terms, the attributes, data structures, or any other programming or structural aspect is not mandatory or significant, and the mechanisms and/or features may have different names, formats, or protocols. Further, the system may be implemented via a combination of hardware and software, or entirely in hardware elements, or entirely in software elements. Also, the particular division of functionality between the various system components described herein is merely exemplary, and not mandatory; functions performed by a single system component may instead be performed by multiple components, and functions performed by multiple components may instead be performed by a single component.

Reference in the specification to “one embodiment” or to “an embodiment” means that a particular feature, structure, or characteristic described in connection with the embodiments is included in at least one embodiment. The appearances of the phrases “in one embodiment” or “in at least one embodiment” in various places in the specification are not necessarily all referring to the same embodiment.

Various embodiments may include any number of systems and/or methods for performing the above-described techniques, either singly or in any combination. Another embodiment includes a computer program product comprising a non-transitory computer-readable storage medium and computer program code, encoded on the medium, for causing a processor in a computing device or other electronic device to perform the above-described techniques.

Some portions of the above may be presented in terms of algorithms and symbolic representations of operations on data bits within a memory of a computing device. These algorithmic descriptions and representations are the means used by those skilled in the data processing arts to most effectively convey the substance of their work to others skilled in the art. An algorithm is here, and generally, conceived to be a self-consistent sequence of steps (instructions) leading to a desired result. The steps are those requiring physical manipulations of physical quantities. Usually, though not necessarily, these quantities take the form of electrical, magnetic or optical signals capable of being stored, transferred, combined, compared and otherwise manipulated. It is convenient at times, principally for reasons of common usage, to refer to these signals as bits, values, elements, symbols, characters, terms, numbers, or the like. Furthermore, it is also convenient at times, to refer to certain arrangements of steps requiring physical manipulations of physical quantities as modules or code devices, without loss of generality.

It should be borne in mind, however, that all of these and similar terms are to be associated with the appropriate physical quantities and are merely convenient labels applied to these quantities. Unless specifically stated otherwise as apparent from the following discussion, it is appreciated that throughout the description, discussions utilizing terms such as “processing” or “computing” or “calculating” or “displaying” or “determining” or the like, refer to the action and processes of a computer system, or similar electronic computing module and/or device, that manipulates and transforms data represented as physical (electronic) quantities within the computer system memories or registers or other such information storage, transmission or display devices.

Certain aspects include process steps and instructions that may be described herein in the form of an algorithm and/or in other terms. It should be noted that the process steps and instructions can be embodied in software, firmware and/or hardware, and when embodied in software, can be downloaded to reside on and be operated from different platforms used by a variety of operating systems.

The present document also relates to an apparatus for performing the operations herein. This apparatus may be specially constructed for the required purposes, or it may comprise a general-purpose computing device selectively activated or reconfigured by a computer program stored in the computing device. Such a computer program may be stored in a computer readable storage medium, such as, but is not limited to, any type of disk including floppy disks, optical disks, CD-ROMs, DVD-ROMs, magnetic-optical disks, read-only memories (ROMs), random access memories (RAMs), EPROMs, EEPROMs, flash memory, solid state drives, magnetic or optical cards, application specific integrated circuits (ASICs), or any type of media suitable for storing electronic instructions, and each coupled to a computer system bus. Further, the computing devices referred to herein may include a single processor or may be architectures employing multiple processor designs for increased computing capability.

The algorithms, processes, and/or displays presented herein are not inherently related to any particular computing device, virtualized system, or other apparatus. Various general-purpose systems may also be used with programs in accordance with the teachings herein, or it may prove convenient to construct more specialized apparatus to perform the required method steps. The required structure for a variety of these systems will be apparent from the description provided herein. In addition, the system and method are not described with reference to any particular programming language. It will be appreciated that a variety of programming languages may be used to implement the teachings described herein, and any references above to specific languages are provided for disclosure of enablement and best mode.

Accordingly, various embodiments include software, hardware, and/or other elements for controlling a computer system, computing device, or other electronic device, or any combination or plurality thereof. Such an electronic device can include, for example, a processor, an input device (such as a keyboard, mouse, touchpad, track pad, joystick, trackball, microphone, and/or any combination thereof), an output device (such as a screen, speaker, and/or the like), memory, long-term storage (such as magnetic storage, optical storage, and/or the like), and/or network connectivity, according to techniques that are well known in the art. Such an electronic device may be portable or non-portable. Examples of electronic devices that may be used for implementing the described system and method include: a smart speaker, mobile phone, personal digital assistant, smartphone, kiosk, server computer, enterprise computing device, desktop computer, laptop computer, tablet computer, consumer electronic device, or the like. An electronic device may use any operating system such as, for example and without limitation: Linux; Microsoft Windows, available from Microsoft Corporation of Redmond, Wash.; Mac OS X, available from Apple Inc. of Cupertino, Calif.; iOS, available from Apple Inc. of Cupertino, Calif.; Android, available from Google, Inc. of Mountain View, Calif.; and/or any other operating system that is adapted for use on the device. In at least one embodiment, the system may be implemented using any suitable computing language, such as for example ReactNative.

While a limited number of embodiments have been described herein, those skilled in the art, having benefit of the above description, will appreciate that other embodiments may be devised. In addition, it should be noted that the language used in the specification has been principally selected for readability and instructional purposes, and may not have been selected to delineate or circumscribe the subject matter. Accordingly, the disclosure is intended to be illustrative, but not limiting, of scope.

What is claimed is:

1. A computer-implemented method for providing personalized health care information and treatment recommendations, comprising:
at a hardware processor, collecting medical information concerning a patient;
at a storage device, storing the collected medical information;
at the hardware processor, retrieving information describing a plurality of treatment options;
at the hardware processor, filtering the treatment options based on the collected medical information, to generate a list of personalized treatment options for the patient; and
at an output device, displaying the personalized treatment options.

2. The method of claim 1, wherein:
retrieving information describing the plurality of treatment options and filtering the treatment options are performed at a server; and
displaying the personalized treatment options comprises transmitting the personalized treatment options to a client device for display thereon.

3. The method of claim 1, wherein collecting medical information concerning a patient comprises at least one selected from the group consisting of:
accessing records containing the patient's medical information; and
receiving user input responding to a questionnaire, wherein the user input comprises the medical information.

4. The method of claim 1, wherein filtering the treatment options comprises determining which treatment options are optimal given the collected medical information.

5. The method of claim 1, wherein filtering the treatment options comprises applying a personalization engine to determine which treatment options are optimal given the collected medical information.

6. A non-transitory computer-readable medium for providing personalized health care information and treatment recommendations, comprising instructions stored thereon, that when performed by a hardware processor, perform the steps of:
collecting medical information concerning a patient;
causing a storage device to store the collected medical information;
retrieving information describing a plurality of treatment options;
filtering the treatment options based on the collected medical information, to generate a list of personalized treatment options for the patient; and
causing an output device to display the personalized treatment options.

7. The non-transitory computer-readable medium of claim 6, wherein:
retrieving information describing the plurality of treatment options comprises causing a server to retrieve the information describing the plurality of treatment options;
filtering the treatment options comprises causing a server to filter the treatment options; and
the output device is at a client device.

8. The non-transitory computer-readable medium of claim 6, wherein collecting medical information concerning a patient comprises at least one selected from the group consisting of:
accessing records containing the patient's medical information; and
receiving user input responding to a questionnaire, wherein the user input comprises the medical information.

9. The non-transitory computer-readable medium of claim 6, wherein filtering the treatment options comprises determining which treatment options are optimal given the collected medical information.

10. The non-transitory computer-readable medium of claim 6, wherein filtering the treatment options comprises applying a personalization engine to determine which treatment options are optimal given the collected medical information.

11. A system for providing personalized health care information and treatment recommendations, comprising:
a hardware processor, configured to:
collect medical information concerning a patient;
retrieve information describing a plurality of treatment options; and
filter the treatment options based on the collected medical information, to generate a list of personalized treatment options for the patient;
a storage device, communicatively coupled to the hardware processor, configured to store the collected medical information; and
at an output device, communicatively coupled to the hardware processor, configured to display the personalized treatment options.

12. The system of claim 11, wherein:
the hardware processor is at a server; and
the output device is at a client device.

13. The system of claim 11, further comprising:
a user input device, communicatively coupled to the hardware processor,
configured to receive user input responding to a questionnaire,
wherein the user input comprises the medical information;
wherein collecting medical information concerning a patient comprises receiving the user input responding to the questionnaire.

14. The system of claim 11, wherein collecting medical information concerning a patient comprises accessing records containing the patient's medical information.

15. The system of claim 11, wherein filtering the treatment options comprises determining which treatment options are optimal given the collected medical information.

16. The system of claim 11, wherein filtering the treatment options comprises applying a personalization engine to determine which treatment options are optimal given the collected medical information.
